(19) United States
(12) Patent Application Publication
(10) Pub. No.: US 2023/0039928 A1
(43) Pub. Date: Feb. 9, 2023

(54) Antisense oligonucleotides for nucleotide deamination in the treatment of Stargardt disease

(71) Applicant: ProQR Therapeutics II B.V., Leiden (NL)

(72) Inventors: Jim Swildens, Leiden (NL); Lenka Van Sint Fiet, Leiden (NL); Tess Hoogeboom, Leiden (NL); Saskia Jacoba Petronella Haast, Leiden (NL)

(21) Appl. No.: 17/788,163

(22) PCT Filed: Dec. 23, 2020
(86) PCT No.: PCT/EP2020/087767

§ 371 Date: Jun. 22, 2022

(30) Foreign Application Priority Data
Dec. 23, 2019 (EP) 19219283.9

(51) Int. Cl. C12N 15/113; C12N 15/90; C12Q 1/6869

(52) U.S. Cl. CPC C12N 15/1138; C12N 15/90; C12Q 1/6869; C12Y 305/04004; C12N 2310/11; C12N 2310/315

(57) ABSTRACT

The invention relates to RNA editing oligonucleotides (EONs) that can bring about specific editing of a target nucleotide (adenosine) in a target RNA molecule in a eukaryotic cell, wherein said oligonucleotide is for use in the treatment of Stargardt disease, and more preferably for the deamination of target adenosines present in the ABCA4 pre-mRNA or ABCA4 mRNA.

### FIELD OF THE INVENTION

The invention relates to the field of medicine and biotechnology. It relates to antisense oligonucleotides that are applicable in the deamination of nucleotides through RNA editing processes using (endogenous) ADAR enzymes by site-specifically targeting alterations (such as G>A mutations) in the ABCA4 (pre-) mRNA of patients suffering from Stargardt disease.

### BACKGROUND OF THE INVENTION

Stargardt disease (STGD or STGD1) is the most common inherited macular dystrophy causing progressive impairment of central vision, with onset typically in childhood or young adulthood, and least frequently in later adulthood, with a better prognosis generally associated with a later onset. The disease has a prevalence of 1 in 8,000-10,000 and has an autosomal recessive mode of inheritance associated with disease-causing mutations in the gene coding for the photoreceptor cell-specific ATP-binding cassette, sub-family A, member 4 protein (ABCA4, sometimes referred to as ABCR). The protein contains 2273 amino acids, is predominantly expressed in the retina (photoreceptor cells and Retinal Pigment Epithelium (RPE)) and localizes to the rims and cone outer segments disks. It is thought to flip N-retinyl-idene-phosphatidylethanolamine from the luminal to the cytosolic face of the photoreceptor disks. Stargardt disease links tightly with a massive deposition of lipofuscin content in the RPE, failure in toxic substance removal and significant loss in photoreceptor cells. A major component of lipofuscin, di-retinoid-pyridinium-ethanolamine is formed when ABCA4 is missing or dysfunctional. Indeed, multiple reports have been published that confirmed that ABCA4 is the gene underlying Stargardt disease, showing a large number (˜1000) of disease-causing variants, of which more than half have been described only once. Biallelic variants in ABCA4 have been identified in approximately 75% Stargardt disease cases and in approximately 30% of patients with autosomal recessive come-rod dystrophy (CRD). Most mutations are missense, followed by nonsense mutations, small insertions/deletions, and mutations affecting RNA splicing. An unusually high proportion of Stargardt disease cases from northern Europe and the United States (˜30%) is the result of a single ABCA4 variant. It is known that the third most frequent ABCA4 variant, c.5461-10T>C present in intron 38, causes a severe form of Stargardt disease due to skipping of exon 39, or skipping of exon 39+exon 40 in the mRNA of ABCA4. The skipping of exon 39 results in a frameshift deletion of 124 nucleotides, whereas the double skip of exon 39 and 40 results in a frameshift deletion of 254 nucleotides. It is estimated that approximately 7000 Stargardt disease patients in the Western world suffer from this mutation.

The three main routes of intervention to treat Stargardt disease are currently stem cell therapy, gene replacement therapy and different pharmaceutical approaches. A relatively new therapeutic development for treating inherited eye diseases is the use of antisense oligonucleotides (AONs), which target the pre-mRNA transcribed from the mutant gene. AONs are generally small polynucleotide molecules (16- to 25-mers) that can interfere with splicing as their sequence is complementary to that of the target pre-mRNA molecule. The envisioned mechanism is such that upon binding of the AON to a target sequence, with which it is complementary, the targeted region within the pre-mRNA interferes with splicing factors which in turn results in altered splicing. Therapeutically, this methodology can be used in two ways: a) to redirect normal splicing of genes in which mutations activate cryptic splice sites and b) to skip exons that carry (protein-truncating) mutations in such a way, that the reading frame of the mRNA remains intact and a (partially or fully) functional protein is made. Both methods have already been successfully applied in patients. With respect to eye diseases, AONs have been shown to be promising for the treatment of Leber's Congenital Amaurosis, or LCA (WO 2012/168435; WO 2013/036105; WO 2016/034680; WO 2016/135334). Further, WO 2016/005514 discloses exon skipping AONs for targeting the USH2A pre-mRNA, directed at skipping of exon 13, exon 50 and PE40, and/or retaining exon 12, for the treatment, prevention, or delay of Usher Syndrome Type II. WO 2015/004133 discloses the use of an AON in stimulating skip of exon 10 from the ABCA4 pre-mRNA, for the treatment of Stargardt disease. WO 2018/189376 discloses antisense oligonucleotides that inhibit skip of exon 39 and exon 40 caused by the c.5461-10T>C mutation. WO 2018/109011 discloses AONs for preventing the inclusion of pseudoexons that are erroneously introduced into the mRNA due to several intronic mutations.

As indicated, when treatment is considered through splice modulation, the prevention of exon skipping, or the prevention of inclusion of a pseudoexon, the resulting mRNA should be in-frame such that the translated protein is functional, or at least partly functional, and not prematurely terminated. However, many mutations exist in the human ABCA4 gene that cannot be solved by splice modulation. For instance, when the resulting transcript will get out of frame, or the resulting protein will lack an essential part required for executing its function. Hence, although a big effort is put into treating Stargardt disease by introducing antisense oligonucleotides that modulate splicing, many Stargardt patients will not benefit from these efforts, because they carry a different kind of mutation in their ABCA4 gene. The c.5882G>A mutation in exon 42 is one that comes to mind and which is one of the more common mutations in ABCA4 in the western world, with an estimated prevalence of 10.000-15.000 patients (Lewis et al. Am J Hum Genet 64:422-434, 1999). This mutation leads to a substitution of the amino acid glycine (G; codon: GGA) at position 1961 of the ABCA4 protein to a glutamic acid (E, codon: GAA). This amino acid is localized in the Nucleotide Binding Domain 2 (NBD2), which is essential for providing the energy for binding of substrate for transport. Exon 42, even though it is in-frame when it would be skipped, encodes for a part of the NBD2 that cannot be disrupted for a properly functioning ABCA4 protein. Biochemical assessment showed that the ABCA4 protein carrying this substitution has reduced ATPase activity (Sun et al. Nature Genetics 26:242-246, 2000), which is also an indication that even the smallest alteration in the NBD2 may cause the protein to become dysfunctional. Other mutations that may be targeted through RNA editing are, and this will be appreciated by the person skilled in the art, mutations that appear in exons that cannot be skipped because the exon is out of frame with its surrounding exons.

### SUMMARY OF THE INVENTION

The present invention relates to an RNA editing oligonucleotide (EON) capable of forming a double stranded complex with a target RNA molecule, wherein the EON when complexed with the target RNA molecule, is able to recruit and complex with an ‘Adenosine Deaminase Acting on RNA’ (ADAR) enzyme, thereby allowing the deamination by the ADAR enzyme of a target adenosine in the target RNA molecule, wherein the EON does not form an internal loop structure and wherein the target RNA molecule is a human ABCA4 pre-mRNA or mRNA, or a part thereof. In one embodiment, the EON comprises a Central Triplet of three sequential nucleotides, wherein the nucleotide directly opposite the target adenosine is the middle nucleotide of the Central Triplet and is a cytidine. Preferably, the ADAR enzyme that is recruited and complexed with the dsRNA complex is ADAR2. In a particularly preferred embodiment one, two or three nucleotides in the Central Triplet comprise a modification, with the proviso that the middle nucleotide does not have a 2′-O-methyl (2′-OMe) or a 2′-methoxyethoxy (2′-MOE) modification in the sugar moiety. It is furthermore preferred that the EON comprises at least one non-naturally occurring internucleoside linkage modification as outlined herein. In a preferred embodiment, the target adenosine is: part of a premature stop codon in the human ABCA4 pre-mRNA or mRNA; any one of the G>A mutations as provided in Table 1; or more preferably the c.5882G>A mutation in exon 42 of the human ABCA4 gene. The invention in a preferred aspect relates to an EON according to the invention, wherein the EON comprises or consists of a sequence selected from the group consisting of: SEQ ID NO:1, 2, 3, 4, 5, 6, 7, 8, 9, and 11. In a preferred embodiment, the EON comprises or consists of a sequence of SEQ ID NO:8 or 9. In an even more preferred embodiment, the EON consists of the sequence of SEQ ID NO:9.

The invention further relates to a pharmaceutical composition comprising an EON according to the invention, and a pharmaceutically acceptable carrier. The invention also relates to a composition comprising a set of two single stranded antisense oligonucleotides (AONs), wherein one AON is an EON according to the invention, and the other AON is the ‘Helper AON’, for use in the deamination of a target adenosine in a human ABCA4 pre-mRNA or mRNA, or a part thereof, wherein the Helper AON is complementary to a stretch of nucleotides in the human ABCA4 pre-mRNA or mRNA that is separate from the stretch of nucleotides that is complementary to the EON, wherein the Helper AON has a length of 16 to 22 nucleotides and the EON has a length of 16 to 22 nucleotides. In a preferred aspect the composition according to the invention comprises as the Helper AON an oligonucleotide that comprises or consists of the sequence of SEQ ID NO:10, and wherein the EON is an oligonucleotide that comprises or consists of the sequence of SEQ ID NO:9.

The invention also relates to an EON or a composition according to the invention for use in the treatment of Stargardt disease. The invention also relates to a method for the deamination of at least one specific target adenosine present in a target RNA molecule in a cell, wherein the target RNA molecule is a human ABCA4 pre-mRNA or mRNA, or a part thereof, and the target adenosine that needs to be deaminated is preferably one of the target adenosines as outlined herein, wherein the method comprises the steps of: providing the cell with an EON or a composition according to the invention; allowing uptake by the cell of the oligonucleotide(s); allowing annealing of the oligonucleotide(s) to the target RNA molecule; allowing a mammalian ADAR enzyme comprising a natural dsRNA binding domain as found in the wild type enzyme to deaminate the target adenosine in the target RNA molecule to an inosine; and optionally identifying the presence of the inosine in the target RNA molecule.

### BRIEF DESCRIPTION OF THE DRAWINGS

FIG. 1 (A) shows the complementarity of EONs referred to as ‘ABCA4-1 to ABCA4-20 (ABCA4-1, 2, 3, 4, and 5 are SEQ ID NO:1, 2, 3, 4, and 5, respectively; ABCA4-6 to 12 are SEQ ID NO:6; ABCA4-13 to 16 are SEQ ID NO:7; ABCA4-17 to 20 are SEQ ID NO:8; all here from 3′ to 5′) used in the examples of the present disclosure. The human ABCA4 target (pre-) mRNA is shown on top (5′ to 3′; SEQ ID NO: 12). The target adenosine A in the target sequence (in exon 42) is given in bold, with the 5′ end of the downstream intron 42 underlined, which is then preceded by the 3′ end of exon 42. ABCA4-13 to 16 are complementary up to the exon42/intron 42 boundary. (B) shows a set the complementarity of EONs referred to as ABCA4-21 to 29 (here from 3′ to 5′). ABCA4-21 to 27 (SEQ ID NO:9) form a set of split EONs each with ABCA4-28 (SEQ ID NO:10), whereas ABCA4-29 (SEQ ID NO:11) is an EON with multiple mismatches. The target adenosine A in the target sequence is given in bold, again with the 5′ end of the downstream intron 42 underlined, preceded by the 3′ end of exon 42. 2′-O-Me RNA is given in lower case. 2′-O-Me nucleotides connected to one another by phosphorothioate linkages are italic and in lower case. DNA nucleotides connected to one another by phosphorothioate linkages are underlined, in upper case and in italics. 2′-MOE modified nucleotides are italic and in upper case. 2′-MOE modified nucleotides connected to one another by phosphorothioate linkages are bold, italic and in upper case. Methylphosphonate (MeP) modified adenosines have a grey box, which is at the linkage position −1 from the middle nucleotide in the Central Triplet (see for the numbering of the linkages: FIG. 5 in WO 2020/201406). The middle nucleotide in the Central Triplet is opposite the target adenosine. 2′-F modified adenosines are bold and have a grey box. Mismatches are boxed in black. ABCA4-5 contains a hairpin sequence (in smaller case).

FIG. 2 shows the results of biochemical assays using ABCA4-1 to 29 from FIG. 1 (except ABCA4-5 and ABCA4-28) in RNA editing reaction applying purified hADAR2, as outlined in the accompanying examples. (A) to (G) each show the results of four different EONs, for clarity.

FIG. 3 shows the pyromark sequencing results of RNA editing on the ABCA4 target sequence using the EONS ABCA4-5, 6, 8, 20, 21+28, 22+28, 23+28, 24+28, and ABCA4-21, 22, 23, 24, and 26 separately in a cell-based assay using midigenes and a plasmid overexpressing ADAR2. WT means the transfection with a midigene carrying the wild type target sequence, whereas ‘mutant’ means a transfection with the mutant midigene alone without ant EON. RT is the reverse transcriptase negative control. All transfections of the EONs were accompanied by a transfection with the mutant midigene.

FIG. 4 shows the ddPCR results of the cell-based assay with the midigenes and over-expression of ADAR2. Using the ddPCR very low RNA editing efficiencies were observed with the EONs that were close to the exon 42 and intron 42 boundary, while the short EONs that were complementary to a sequence further away from the exon/intron boundary, showed significant levels of RNA editing.

FIG. 5 shows the result of an exon skip checking experiment, that reveals that transfections of the EONs ABCA4-5 to 16, as well as ABCA-29 results in a certain level of exon 42 skip, which explains the lower RNA editing efficiency observed with these EONs (that are closer to the exon 42/intron 42 boundary) in the pyromark and ddPCR sequence results.

### DETAILED DESCRIPTION

The inventors of the present invention have sought for other means than splice modulation to correct defects in the (pre-) mRNA within ABCA4. The inventors contemplated using another type of RNA repair, often referred to as ‘RNA editing’. RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms such as Caenorhabditis elegans, to humans. Examples of RNA editing are adenosine (A) to inosine (I) and cytidine (C) to uridine (U) conversions through enzymes called adenosine deaminase and cytidine deaminase, respectively. The most extensively studied RNA editing system is the adenosine deaminase enzyme, which is a multi-domain protein, comprising a recognition domain and a catalytic domain. The recognition domain recognizes a specific double-stranded RNA (dsRNA) sequence and/or conformation, whereas the catalytic domain converts an adenosine into an inosine in a nearby, predefined position in the target RNA, by deamination of the nucleobase. Inosine is read as guanosine by the translational machinery of the cell, meaning that, if an edited adenosine is in a coding region of an mRNA or pre-mRNA, it can recode the protein sequence. RNA editing by adenosine deamination is therefore a perfect way of repairing G>A mutations, but also other mutations wherein conversion from A to G would allow the generation of a functional protein. The adenosine deaminases are part of a family of enzymes referred to as Adenosine Deaminases acting on RNA (ADAR), including human deaminases hADAR1, hADAR2 and hADAR3.

The use of oligonucleotides to edit target RNA applying adenosine deaminase is known in the art. Montiel-Gonzalez et al. (Proc Natl Acad Sci USA 2013, 110(45):18285-18290) described the editing of a target RNA using a genetically engineered fusion protein, comprising an adenosine deaminase domain of the hADAR2 protein, fused to a bacteriophage lambda N protein, which recognises the boxB RNA hairpin sequence. A disadvantage of this method in a therapeutic setting is the need for the fusion protein. It requires cells to be either transduced with the fusion protein, which is a major hurdle, or that target cells are transfected with a nucleic acid construct encoding the engineered adenosine deaminase fusion protein for expression. Vogel et al. (2014. Angewandte Chemie Int Ed 53:267-271) disclosed editing of RNA coding for eCFP and Factor V Leiden, using a benzylguanosine substituted guide RNA and a genetically engineered fusion protein, comprising the adenosine deaminase domains of ADAR1 or 2 (lacking the dsRNA binding domains) genetically fused to a SNAP-tag domain (an engineered O6-alkylguanosine-DNA-alkyl transferase). This system suffers from similar drawbacks as the engineered ADARs described by Montiel-Gonzalez et al. (2013). Woolf et al. (1995. Proc Natl Acad Sci USA 92:8298-8302) disclosed a simpler approach, using relatively long single-stranded antisense RNA oligonucleotides (25-52 nucleotides in length) wherein the longer oligonucleotides (34-mer and 52mer) could promote editing of the target RNA by endogenous ADAR because of the double-stranded nature of the target RNA and the hybridizing oligonucleotide, but only appeared to function in cell extracts or in amphibian (Xenopus) oocytes by microinjection, and suffered from severe lack of specificity: nearly all adenosines in the target RNA strand that was complementary to the antisense oligonucleotide were edited. Woolf et al. (1995) did not achieve deamination of a specific target adenosine in the target RNA sequence, because nearly all adenosines opposite an unmodified nucleotide in the antisense oligonucleotide were edited through a process sometimes referred to as ‘promiscuous editing’. WO2016/097212 discloses RNA editing oligonucleotides, characterized by a sequence that is complementary to a target RNA sequence (‘targeting portion’) and by the presence of a stem-loop structure (‘recruitment portion’). WO2017/220751 discloses RNA editing oligonucleotides without a recruitment portion, but with a strand of nucleotides that are complementary to a target region, for the specific editing of a single adenosine, and wherein the oligonucleotide comprises one or more mismatches, wobbles and/or bulges in combination with specific chemical modifications. Very specific locations of specific chemical modifications in such RNA editing oligonucleotides were further disclosed in WO2018/041973, WO2019/158475, and WO2019/219581. WO2019/005884 discloses a system for deamination of target adenosines in which a targeting system comprises a targeting domain (a CRISPR system comprising a CRISPR effector protein (such as Cas13) and a guide molecule that is generally an oligonucleotide with a sequence that is complementary to the target sequence) linked to an adenosine deaminase, or a catalytic domain thereof. The present invention relates to ‘naked’ oligonucleotides (as is), or that are expressed from a (viral) vector), but that in either way does not comprise a targeting domain that generates a loop structure for complexing with an effector protein such as those of the CRISPR system. Where RNA-editing oligonucleotides as disclosed in WO2016/097212 and WO2019/005884 with their internal loop structures that recruit or are linked to effector proteins may be considered as first-generation RNA editing oligonucleotides, the present invention relates to RNA editing oligonucleotides that do not contain such an internal loop structure and may therefore be regarded as the second-generation RNA editing oligonucleotides. WO2019/005884 discloses RNA editing oligonucleotides for use in targeting G>A mutations in the USH2A gene, that causes Usher Syndrome type II, a degenerative disease of the retina and inner ear. WO2019/005884 discloses RNA editing oligonucleotides that form a loop structure, and that are also complexed to a (mutated) ADAR enzyme. The present invention relates to RNA editing oligonucleotides that do not comprise a sequence that forms a loop structure, and that are not complexed (covalently or non-covalently) to an ADAR enzyme, or a mutated ADAR enzyme, but rather makes use of endogenous deaminase enzymes such as ADAR, which is already present in the target cell. The RNA editing oligonucleotides of the present invention recruit such enzymes after administration to the cell or the tissue, inside the cell, but are also capable of recruiting ADAR enzymes in biochemical assays as shown in the accompanying example(s). In a preferred embodiment, the deaminase is an ADAR enzyme, more preferably ADAR2, that is already present at endogenous levels inside the target cell and does not need to be co-administered, such as through an expression vector, or otherwise.

Most of the RNA editing prior art relates to the general applicability of this phenomenon for any type of disease or genetic disorder in which a specific target adenosine should be edited to an inosine to restore translation (where the adenosine was part of a stop codon), and/or to repair the RNA when the adenosine was part of a codon that altered the protein and caused the genetic disease. The documents in the prior art did not specifically reveal the application of RNA editing oligonucleotides in eye diseases such as Stargardt disease, in which genetic alterations are the cause of the disorder, or how this specifically should be performed. In contrast, a lot of prior art has accumulated that reveals the usefulness of antisense oligonucleotides in downregulating protein expression, or that influence splicing (e.g., see WO2012/168435, WO2013/036105, WO2016/005514, WO2016/034680, WO2016/138353, WO2016/135334, WO2017/060317, WO2017/186739, WO2018/055134, WO2015/004133, WO2018/189376, WO2018/109011, and U.S. Pat. No. 9,353,371). To the best of the knowledge of the inventors of the present invention the use of RNA editing oligonucleotides has not been published for the purpose to deaminate specific adenosines in genes causing eye defects, and applying such RNA editing oligonucleotides in the treatment of eye disorders, more specifically in Stargardt disease.

The present invention relates to RNA editing oligonucleotides (herein generally abbreviated to “EONs”) and their use in the treatment of eye disease, particularly Stargardt disease. The EONs of the present invention target specific adenosines in the ABCA4 pre-mRNA or mRNA and deaminate these to inosines, read as guanosines in translation. It is noted that the adenosine (A) itself does not always have to be the mutation causing Stargardt disease but may for instance be part of a premature termination codon that is the cause of the disease (since, for instance a shorter ABCA4 protein product is produced), and in which for instance the guanosine (G) or the thymidine (T) is the real mutation. Deamination of the adenosine to an inosine may result in a wild type protein, or in a protein with an altered amino acid instead of the stop codon (and altered in respect of the original codon of the wild type mRNA) but should allow continued translation.

The invention relates to an EON capable of forming a double stranded complex with a target RNA molecule, wherein the EON when complexed with the target RNA molecule, is able to recruit and complex with an ‘Adenosine Deaminase Acting on RNA’ (ADAR) enzyme, thereby allowing the deamination by the ADAR enzyme of a target adenosine in the target RNA molecule, wherein the EON does not form an internal loop structure and wherein the target RNA molecule is a human ABCA4 pre-mRNA or mRNA, or a part thereof. It is to be noted that the ADAR, preferably ADAR2, is not complexed with the EON when the EON is administered, to a cell, tissue or (human) subject. It is to be noted that the EON is delivered to the cell as is (naked), or through expression from a viral vector or other expression vector. Once in the cell, the EON targets the ABCA4 target pre-mRNA or mRNA, and hybridizes with its target. This double stranded complex, due to its mismatches and selective modifications and content of the EON is then able to attract (or recruit) an endogenous ADAR enzyme, which then subsequently deaminates the target adenosine, that is opposite the central nucleotide in the Central Triplet, preferably a cytidine. This allows for specific RNA editing of adenosines in the human ABCA4 pre-mRNA or mRNA. The EON of the present invention is not bound to or (non-)covalently attached to any proteins before it enters the cell, when administered in a ‘naked’ form. It also does not form an internal loop structure to bind ADAR, or any other effector proteins, such as seen in the art for the CRISPR/Cas system. The EON of the present invention comprises a Central Triplet of three sequential nucleotides, wherein the nucleotide directly opposite the target adenosine is the middle nucleotide of the Central Triplet and is preferably a cytidine. Preferably, the ADAR enzyme that is attracted by the dsRNA complex is ADAR2. As shown in the accompanying example(s) the person skilled in the art can set up a biochemical assay that is representative for the in vivo situation in the sense that the EON is brought into contact with its target sequence and recruit ADAR enzyme within the assay and bring about RNA editing of a specific target adenosine in the ABCA4 target molecule. In a preferred embodiment one, two or three nucleotides in the Central Triplet comprise a modification, with the proviso that the middle nucleotide does not have a 2′-O-methyl (2′-OMe) or a 2′-methoxyethoxy (2′-MOE) modification in the sugar moiety. In another preferred embodiment, the modification of the nucleotide in the Central Triplet is selected from the group consisting of deoxyribose (DNA), Unlocked Nucleic Acid (UNA) and 2′-fluororibose. As outlined further herein, DNA is then considered to be a chemical derivative of RNA. Most (but in some embodiments certainly not all) nucleotides within an EON of the present invention is RNA, that may be modified with non-naturally occurring substituents as detailed further herein. An EON of the present invention preferably comprises at least one non-naturally occurring internucleoside linkage modification selected from the group consisting of: phosphorothioate, chirally pure phosphorothioate, Rp phosphorothioate, Sp phosphorothioate, phosphorodithioate, phosphonoacetate, thophosphonoacetate, phosphonacetamide, thiophosphonacetamide, phosphorothioate prodrug, S-alkylated phosphorothioate, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphorylguanidine, methyl sulfonylphosphoroamidate, phosphoramidite, phosphonamidite, N3′→P5′ phosphoramidate, N3′→P5′ thiophosphoramidate, phosphorodiamidate, phosphorothiodiamidate, sulfamate, dimethylenesulfoxide, sulfonate, triazole, oxalyl, carbamate, methyleneimino, thioacetamido, and their derivatives. Most preferred is the use of phosphorothioate linkages, although the EON does not have to be fully phosphorothioated. For instance, in particular embodiment, at least the two, three, four, five, or six terminal nucleotides of the 5′ and 3′ terminus of the EON are linked with phosphorothioate linkages, preferably wherein the terminal five nucleotides at the 5′ and 3′ terminus are linked with phosphorothioate linkages. Further nucleotides more towards the Central Triplet may also be connected by a non-naturally occurring linkage, such as phosphorothioates. In a preferred embodiment, the EON is chemically modified to render the EON stable towards breakdown by the cellular RNases and other environmental circumstances in vivo. For this, it is preferred that one or more nucleotides in the EON outside the Central Triplet comprise a mono- or disubstitution at the 2′, 3′ and/or 5′ position of the sugar, selected from the group consisting of: —OH; —F; substituted or unsubstituted, linear or branched lower (CI-C10) alkyl, alkenyl, alkynyl, alkaryl, allyl, or aralkyl, that may be interrupted by one or more heteroatoms; O-, S-, or N-alkyl; O-, S-, or N-alkenyl; O-, S-, or N-alkynyl; O-, S-, or N-allyl; O-alkyl-O-alkyl; -methoxy; -aminopropoxy; -methoxyethoxy; -dimethylamino oxyethoxy; and -dimethylaminoethoxyethoxy. Other possible chemical modifications that may be introduced into the EON of the present invention, especially then of the sugar, the base and linker are discussed further herein.

RNA editing according to the system as outlined by the present invention, preferably targets a single adenosine in a target molecule (a pre-mRNA or mRNA transcribed of the human ABCA4 gene). Preferably, the target adenosine is part of a premature stop codon in the human ABCA4 pre-mRNA or mRNA. In another preferred embodiment, the target adenosine is any one of the G>A mutations as provided in Table 1. More preferably the target adenosine is the c.5882G>A mutation in exon 42 of the human ABCA4 gene. The nucleotide at the 3′ side of this G>A mutation is also an adenosine in the human ABCA4 gene. That second adenosine may be co-edited with the target adenosine, because a GGA (wt; glycine) to GAA (mutation; glutamic acid) and repaired to GGA or GGG would both result in a glycine at that codon.

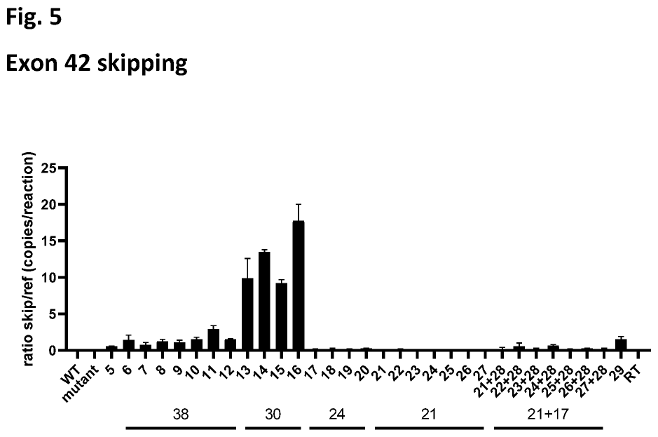

The invention further relates to an EON that targets the (pre-) mRNA sequence of SEQ ID NO:12, or a part thereof that includes the G>A mutation. Preferably, the invention relates to an EON that comprises or consists of the sequence selected from the group consisting of: SEQ ID NO:1, 2, 3, 4, 5, 6, 7, 8, 9, and 11. In a preferred embodiment, the EON comprises or consists of a sequence of SEQ ID NO:8 or 9. In an even more preferred embodiment, the EON consists of the sequence of SEQ ID NO:9. In another preferred embodiment, the EON according to the invention comprises at least one methylphosphonate (MeP, or MP) linkage, preferably at linkage position −1, following the nucleotide and linkage numbering as disclosed in FIG. 5 of WO 2020/201406.

The invention further relates to a pharmaceutical composition comprising an EON according to the invention, and a pharmaceutically acceptable carrier. The invention also relates to a composition comprising a set of two single stranded antisense oligonucleotides (AONs), wherein one AON is an EON according to the invention, and the other AON is the ‘Helper AON’, for use in the deamination of a target adenosine in a human ABCA4 pre-mRNA or mRNA, or a part thereof, wherein the Helper AON is complementary to a stretch of nucleotides in the human ABCA4 pre-mRNA or mRNA that is separate from the stretch of nucleotides that is complementary to the EON, wherein the Helper AON has a length of 16 to 22 nucleotides and the EON has a length of 16 to 22 nucleotides. Preferably, the EON comprises or consists of a sequence of: SEQ ID NO:9, and the Helper AON comprises or consists of a sequence according to SEQ ID NO:10.

The invention further relates to an EON according to the invention for use in the treatment of Stargardt disease.

The invention further relates to the use of an EON according to the invention for the manufacture of a medicament for use in the treatment, amelioration, prevention or slowing down the progression of Stargardt disease.

The invention further relates to a method for the deamination of at least one specific target adenosine present in a target RNA molecule in a cell, wherein the target RNA molecule is a human ABCA4 pre-mRNA or mRNA, or a part thereof, the method comprising the steps of: providing the cell with an EON according to the invention; allowing uptake by the cell of the EON; allowing annealing of the EON to the target RNA molecule; allowing a mammalian ADAR enzyme comprising a natural dsRNA binding domain as found in the wild type enzyme to deaminate the target adenosine in the target RNA molecule to an inosine; and optionally identifying the presence of the inosine in the target RNA molecule. The optional step preferably comprises: (i) sequencing the target RNA molecule; (ii) assessing the presence of a functional, elongated, full length and/or wild type ABCA4 protein; (iii) assessing whether splicing of the pre-mRNA was modulated by the deamination; or (iv) using a functional read-out, wherein the target RNA after the deamination encodes a functional, full length, elongated and/or wild type ABCA4 protein.

The invention further relates to a method of treating, ameliorating, preventing and/or slowing down the progression of Stargardt disease in a human subject in need thereof that is suffering from, or is at risk of suffering from, Stargardt disease, comprising the step of administering an EON according to the invention, or a pharmaceutical composition according to the invention, to the human subject. In a preferred embodiment, the EON is administered to the human subject in need thereof, by intravitreal administration, preferably through direct injection of the naked (and chemically modified) EON, and allowing the targeting of a mutated ABCA4 pre-mRNA or mutated ABCA4 mRNA in a retinal cell, preferably a photoreceptor cell or a cell of the RPE to alleviate the disease-causing actions of the mutant ABCA4 protein, by allowing the translation of the edited (and then preferably mostly wild type) ABCA4 mRNA.

An EON of the present invention does not comprise a recruitment portion as described in WO2016/097212. The EONs of the present invention do not comprise a portion that can form an intramolecular stem-loop structure. The EONs of the present invention are shorter than those forming a (ADAR recruiting) loop structure, which makes them cheaper to produce, easier to use and easier to manufacture. Furthermore, they are likely to enter cells more efficiently than longer oligonucleotides and are less prone to degradation. WO2017/220751 and WO2018/041973 disclose EONs that are complementary to a target RNA for deaminating a target adenosine present in a target RNA sequence to which the EON is complementary, but also lacked a recruitment portion while still being capable of harnessing ADAR enzymes present in the cell to edit the target adenosine. The present invention makes use of that knowledge and aims to solve the problem of targeting Stargardt disease mutations for which exon skipping is not the preferred therapeutic option, or where an alternative approach would be sought.

An EON of the present invention comprises one or more nucleotides with one or more non-naturally occurring sugar modifications. Thereby, a single nucleotide of the EON can have one, or more than one such sugar modification. Within the EON, one or more nucleotide(s) can have such sugar modification(s). It is also an aspect of the invention that the nucleotide within the EON of the present invention that is opposite to the nucleotide that needs to be edited does not contain a 2′-O-methyl (2′-OMe) or a 2′-methoxyethoxy (2′-MOE) modification. Often the nucleotides that are directly 3′ and 5′ of this nucleotide (the ‘neighbouring nucleotides’) in the EON also lack such a chemical modification, although it is not mandatory that both neighbouring nucleotides should not contain a 2′-O-alkyl group (such as a 2′-OMe). Either one, both neighbouring nucleotides, or all three nucleotides of the ‘Central Triplet’ may carry 2′-OH.

The skilled person knows that an oligonucleotide, such as an RNA oligonucleotide, generally consists of repeating monomers. Such a monomer is most often a nucleotide or a nucleotide analogue. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. For severe modifications, the original pentose sugar might be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine or uracil, or a derivative thereof. Cytosine, thymine, and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked monomers of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate, such a moiety is still referred to as the backbone linkage of the monomer. This is referred to as a “backbone linkage modification”. In general terms, the backbone of an oligonucleotide comprises alternating scaffolds and backbone linkages.

In one embodiment, the nucleobase in an EON of the present invention is adenine, cytosine, guanine, thymine, or uracil. In another embodiment, the nucleobase is a modified form of adenine, cytosine, guanine, or uracil. In another embodiment, the modified nucleobase is hypoxanthine (the nucleobase in inosine), pseudouracil, pseudocytosine, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine, 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methyl ribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar. The term ‘nucleoside’ refers to the nucleobase linked to the (deoxy)ribosyl sugar. The term ‘nucleotide’ refers to the respective nucleobase-(deoxy)ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group, well known in the art), a nucleotide including a linker comprising a phosphodiester, phosphotriester, phosphoro(di)thioate, methylphosphonates, phosphoramidate linkers, and the like. Sometimes the terms adenosine and adenine, guanosine and guanine, cytosine and cytidine, uracil and uridine, thymine and thymidine, inosine and hypoxanthine, are used interchangeably to refer to the corresponding nucleobase, nucleoside or nucleotide. Sometimes the terms nucleobase, nucleoside and nucleotide are used interchangeably, unless the context clearly requires differently.

In one embodiment, an EON of the present invention comprises a 2′-substituted phosphorothioate monomer, preferably a 2′-substituted phosphorothioate RNA monomer, a 2′-substituted phosphate RNA monomer, or comprises 2′-substituted mixed phosphate/phosphorothioate monomers. It is noted that DNA is considered as an RNA derivative in respect of 2′ substitution. An EON of the present invention comprises at least one 2′-substituted RNA monomer connected through or linked by a phosphorothioate or phosphate backbone linkage, or a mixture thereof. The 2′-substituted RNA preferably is 2′-F, 2′-H (DNA), 2′-O-Methyl or 2′-O-(2-methoxyethyl). The 2′-O-Methyl is often abbreviated to “2′-OMe” and the 2′-O-(2-methoxyethyl) moiety is often abbreviated to “2′-MOE”. More preferably, the 2′-substituted RNA monomer in the EON of the present invention is a 2′-OMe monomer, except for the monomer opposite the target adenosine, as further outlined herein, which should not carry a 2′-OMe substitution. In a preferred embodiment of this aspect is provided an EON according to the invention, wherein the 2′-substituted monomer can be a 2′-substituted RNA monomer, such as a 2′-F monomer, a 2′-NH₂ monomer, a 2′-H monomer (DNA), a 2′-O-substituted monomer, a 2′-OMe monomer or a 2′-MOE monomer or mixtures thereof. Preferably, the monomer opposite the target adenosine is a 2′-H monomer (DNA) but may also be a monomer that allows deamination of the target adenosine, other than a 2′-OMe monomer. Preferably, any other 2′-substituted monomer within the EON is a 2′-substituted RNA monomer, such as a 2′-OMe RNA monomer or a 2′-MOE RNA monomer, which may also appear within the EON in combination.

Throughout the application, a 2′-OMe monomer within an EON of the present invention may be replaced by a 2′-OMe phosphorothioate RNA, a 2′-OMe phosphate RNA or a 2′-OMe phosphate/phosphorothioate RNA. Throughout the application, a 2′-MOE monomer may be replaced by a 2′-MOE phosphorothioate RNA, a 2′-MOE phosphate RNA or a 2′-MOE phosphate/phosphorothioate RNA. Throughout the application, an oligonucleotide consisting of 2′-OMe RNA monomers linked by or connected through phosphorothioate, phosphate or mixed phosphate/phosphorothioate backbone linkages may be replaced by an oligonucleotide consisting of 2′-OMe phosphorothioate RNA, 2′-OMe phosphate RNA or 2′-OMe phosphate/phosphorothioate RNA. Throughout the application, an oligonucleotide consisting of 2′-MOE RNA monomers linked by or connected through phosphorothioate, phosphate or mixed phosphate/phosphorothioate backbone linkages may be replaced by an oligonucleotide consisting of 2′-MOE phosphorothioate RNA, 2′-MOE phosphate RNA or 2′-MOE phosphate/phosphorothioate RNA.

In addition to the specific preferred chemical modifications at certain positions in compounds of the invention, compounds of the invention may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-O-methyl, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-O-(haloalkyl)methyl such as 2′-O-(2-chloroethoxy)methyl (MCEM), 2′-0-(2,2-dichloroethoxy)methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DOME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-0,4′-C) constrained ethyl (cEt) BNA monomer, a (2′-0,4′-C) constrained methoxyethyl (cMOE) BNA monomer, a 2′,4′-BNA^{NC}(NH) monomer, a 2′,4′-BNA^{NC}(NMe) monomer, a 2′,4′-BNA^{NC}(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All of these modifications are known to the person skilled in the art.

A “backbone modification” indicates the presence of a modified version of the ribosyl moiety (“scaffold modification”), as indicated above, and/or the presence of a modified version of the phosphodiester as naturally occurring in RNA (“backbone linkage modification”). Examples of internucleoside linkage modifications are phosphorothioate (PS), chirally pure phosphorothioate, Rp phosphorothioate, Sp phosphorothioate, phosphorodithioate (PS2), phosphonoacetate (PACE), thophosphonoacetate, phosphonacetamide (PACA), thiophosphonacetamide, phosphorothioate prodrug, S-alkylated phosphorothioate, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphoryl guanidine (PGO), methylsulfonyl phosphoroamidate, phosphoramidite, phosphonamidite, N3′→P5′ phosphoramidate, N3′→P5′ thiophosphoramidate, phosphorodiamidate, phosphorothiodiamidate, sulfamate, dimethylenesulfoxide, sulfonate, triazole, oxalyl, carbamate, methyleneimino (MMI), and thioacetamido (TANA); and their derivatives.

Preferred EONs of the present invention do not include a 5′-terminal O6-benzylguanosine or a 5′-terminal amino modification and are not covalently linked to a SNAP-tag domain (an engineered O6-alkylguanosine-DNA-alkyl transferase). In one embodiment, an EON of the present invention comprises 0, 1, 2 or 3 wobble base pairs with the target sequence, and/or 0, 1, 2, or 3 mismatches with the target RNA sequence, wherein a single mismatch may comprise multiple sequential nucleotides. Similarly, a preferred EON of the present invention does not include a boxB RNA hairpin sequence. An EON according to the present invention can utilise endogenous cellular pathways and naturally available ADAR enzymes to specifically edit a target adenosine in a target RNA sequence. An EON of the invention is capable of recruiting ADAR and complex with it and then allow the deamination of a (single) specific target adenosine nucleotide in a target RNA sequence. Ideally, only one adenosine is deaminated. Alternatively, 1, 2, or 3 adenosine nucleotides are deaminated, for instance when target adenosines are in proximity of each other. For example, when the mutation is an alteration from a wild type GGA (glycine) codon to a mutant GAA (glutamic acid) codon, deamination of both adenosines would result in GGG, which also encodes a glycine. An EON of the invention, when complexed to ADAR, preferably deaminates a single target adenosine.

Analysis of natural targets of ADAR enzymes has indicated that these generally include mismatches between the two strands that form the RNA helix edited by ADAR1 or 2. It has been suggested that these mismatches enhance the specificity of the editing reaction (Stefl et al. 2006. Structure 14(2):345-355; Tian et al. 2011. Nucleic Acids Res 39(13):5669-5681). Characterization of optimal patterns of paired/mismatched nucleotides between the EONs and the target RNA also appears crucial for development of efficient ADAR-based EON therapy.

An EON of the present invention makes use of specific nucleotide modifications at predefined spots to ensure stability as well as proper ADAR binding and activity. These changes may vary and may include modifications in the backbone of the EON, in the sugar moiety of the nucleotides as well as in the nucleobases or the phosphodiester linkages, as outlined in detail above. They may also be variably distributed throughout the sequence of the EON. Specific modifications may be needed to support interactions of different amino acid residues within the RNA-binding domains of ADAR enzymes, as well as those in the deaminase domain. For example, phosphorothioate linkages between nucleotides or 2′-OMe or 2′-MOE modifications may be tolerated in some parts of the EON, while in other parts they should be avoided so as not to disrupt crucial interactions of the enzyme with the phosphate and 2′-OH groups. Specific nucleotide modifications may also be necessary to enhance the editing activity on substrate RNAs where the target sequence is not optimal for ADAR editing. Previous work has established that certain sequence contexts are more amenable to editing. For example, the target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42(10):e87). The structural analysis of ADAR2 deaminase domain hints at the possibility of enhancing editing by careful selection of the nucleotides that are opposite to the target trinucleotide. For example, the 5′-CAA-3′ target sequence, paired to a 3′-GCU-S′ sequence on the opposing strand (with the A-C mismatch formed in the middle), is disfavored because the guanosine base sterically clashes with an amino acid side chain of ADAR2.

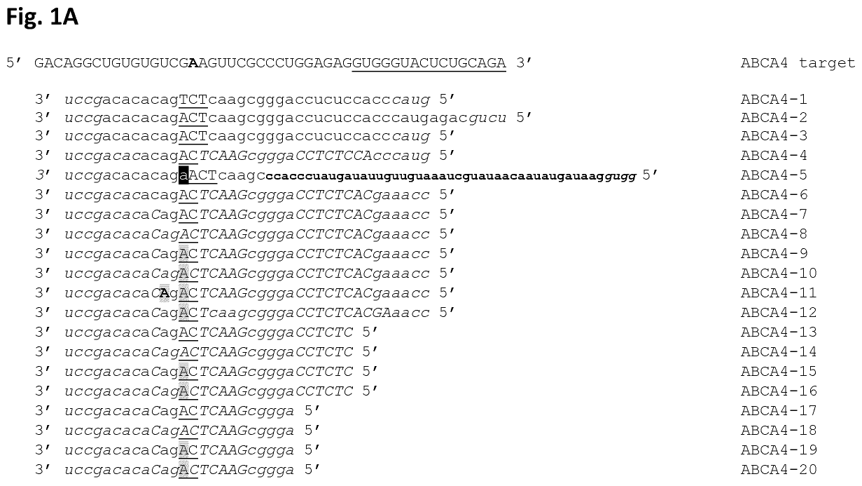

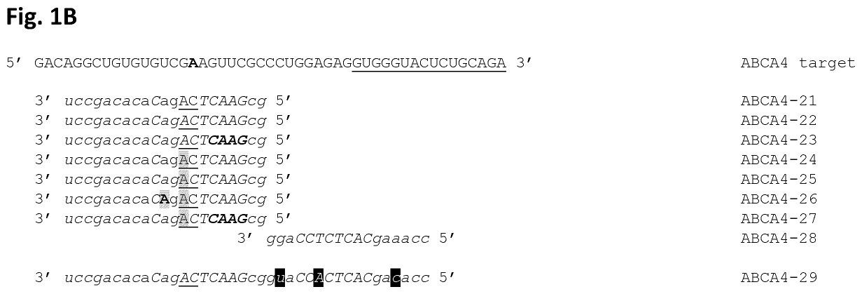

The “Central Triplet” as used herein are the three nucleotides opposite the target adenosine in the target RNA, wherein the middle nucleotide in the Central Triplet is directly opposite the target adenosine. The Central Triplet does not have to be in the centre of the EON, as it may be located more to the 3′ as well as to the 5′ end of the EON, whatever is preferred for a certain target. The term “Central” in this aspect has therefore more the meaning of the triplet that is in the centre of catalytic activity when it comes to chemical modifications and targeting the target adenosine. It should also be noted that the EONs are sometimes depicted from 3′ to 5′, especially when the target sequence is shown from 5′ to 3′ (see for instance FIG. 1). However, whenever herein the order of nucleotides within the EON is discussed it is always from 5′ to 3′ of the EON. The position can also be expressed in terms of a particular nucleotide within the EON while still adhering to the 5′ to 3′ directionality, in which case other nucleotides 5′ of the said nucleotide are marked as negative positions and those 3′ of it as positive positions.

As outlined herein, the nucleotides outside the Central Triplet are often 2′-OMe or 2′-MOE modified. However, this is not a strict requirement of the EONs of the present invention. The use of these 2′ substitutions assures a proper stability of those parts of the EON, but other modifications may be applied as well.

An EON according to the invention may be indirectly administrated using suitable means known in the art. It may for example be provided to an individual or a cell, tissue, or organ of said individual in the form of an expression vector wherein the expression vector encodes a transcript comprising said oligonucleotide. The expression vector is preferably introduced into a cell, tissue, organ, or individual via a gene delivery vehicle. In a preferred embodiment, there is provided a viral-based expression vector comprising an expression cassette or a transcription cassette that drives expression or transcription of an EON as identified herein. Accordingly, the invention provides a viral vector that can express an EON according to the invention (then without non-natural chemical modifications) when placed under conditions conducive to expression of the EON. A cell can be provided with an EON by plasmid-derived EON expression or viral expression provided by adenovirus- or adeno-associated virus-based vectors. Expression may be driven by a polymerase II-promoter (Pol II) such as a U7 promoter or a polymerase III (Pol III) promoter, such as a U6 RNA promoter. A preferred delivery vehicle is AAV, or a retroviral vector such as a lentivirus vector and the like. Also, plasmids, artificial chromosomes, plasmids usable for targeted homologous recombination and integration in the human genome of cells may be suitably applied for delivery of an EON as defined herein. Preferred for the current invention are those vectors wherein transcription is driven from Pol III promoters, and/or wherein transcripts are in the form fusions with U1 or U7 transcripts, which yield good results for delivering small transcripts. It is within the skill of the artisan to design suitable transcripts. Preferred are Pol III driven transcripts, preferably, in the form of a fusion transcript with an U1 or U7 transcript, known to the person skilled in the art.

Typically, when the EON is delivered by a viral vector, it is in the form of an RNA transcript that comprises the sequence of an oligonucleotide according to the invention in a part of the transcript. The resulting EON that is active in the cell is then not chemically modified because it is naturally expressed, while an EON that is manufactured in a ‘naked’ form (=without the use of a plasmid or viral vector expressing the EON) may comprise single or multiple non-naturally occurring modifications. An AAV vector according to the invention is a recombinant AAV vector and refers to an AAV vector comprising part of an AAV genome comprising an encoded EON according to the invention encapsidated in a protein shell of capsid protein derived from an AAV serotype. Part of an AAV genome may contain the inverted terminal repeats (ITR) derived from an adeno-associated virus serotype, such as AAV1, AAV2, AAV3, AAV4, AAV5, AAV6, AAV7, AAV8, AAV9 and others. Protein shell comprised of capsid protein may be derived from an AAV serotype such as AAV1, 2, 3, 4, 5, 6, 7, 8, 9 and others. A protein shell may also be named a capsid protein shell. An AAV vector may have one or preferably all wild type AAV genes deleted but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, if they remain functional. In this context, functionality refers to the ability to direct packaging of the genome into the capsid shell and then allow for expression in the host cell to be infected or target cell. In the context of the invention a capsid protein shell may be of a different serotype than the AAV vector genome ITR. An AAV vector according to present the invention may thus be composed of a capsid protein shell, i.e., the icosahedral capsid, which comprises capsid proteins (VP1, VP2, and/or VP3) of one AAV serotype, e.g. AAV serotype 2, whereas the ITRs sequences contained in that AAV2 vector may be any of the AAV serotypes described above, including an AAV2 vector. An “AAV2 vector” thus comprises a capsid protein shell of AAV serotype 2, while e.g., an “AAV5 vector” comprises a capsid protein shell of AAV serotype 5, whereby either may encapsidate any AAV vector genome ITR according to the invention. Preferably, a recombinant AAV vector according to the invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the invention comprises a capsid protein shell of AAV serotype 2 and the AAV genome or ITRs present in said vector are derived from AAV serotype 5; such vector is referred to as an AAV 2/5 vector. More preferably, a recombinant AAV vector according to the invention comprises a capsid protein shell of AAV serotype 2 and the AAV genome or ITRs present in said vector are derived from AAV serotype 8; such vector is referred to as an AAV 2/8 vector. More preferably, a recombinant AAV vector according to the invention comprises a capsid protein shell of AAV serotype 2 and the AAV genome or ITRs present in said vector are derived from AAV serotype 9; such vector is referred to as an AAV 2/9 vector. More preferably, a recombinant AAV vector according to the invention comprises a capsid protein shell of AAV serotype 2 and the AAV genome or ITRs present in said vector are derived from AAV serotype 2; such vector is referred to as an AAV 2/2 vector. A nucleic acid molecule encoding an EON according to the invention represented by a nucleic acid sequence of choice is preferably inserted between the AAV genome or ITR sequences as identified above, for example an expression construct comprising an expression regulatory element operably linked to a coding sequence and a 3′ termination sequence. “AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art. The AAV helper functions can be supplied on an AAV helper construct, which may be a plasmid.

Introduction of the helper construct into the host cell can occur e.g. by transformation, transfection, or transduction prior to or concurrently with the introduction of the AAV genome present in the AAV vector as identified herein. The AAV helper constructs of the invention may thus be chosen such that they produce the desired combination of serotypes for the AAV vector's capsid protein shell on the one hand and for the AAV genome present in said AAV vector replication and packaging on the other hand. “AAV helper virus” provides additional functions required for AAV replication and packaging.

Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456. Preferably, an AAV genome as present in a recombinant AAV vector according to the invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art. A preferred AAV vector according to the invention is an AAV vector, preferably an AAV2/5, AAV2/8, AAV2/9 or AAV2/2 vector, expressing an EON according to the invention.

Whenever reference is made to an ‘antisense oligonucleotide’ (‘AON’), an ‘RNA editing oligonucleotide’ (EON), an ‘oligonucleotide’, or an ‘oligo’ then both oligoribonucleotides and deoxyoligoribonucleotides are meant unless the context dictates otherwise. Whenever reference is made to an ‘oligoribonucleotide’ it may comprise the ribonucleosides adenosine (A), guanosine (G), cytidine (C), 5-methylcytidine (m⁵C), uridine (U), 5-methyluridine (m⁵U) or inosine (I). Whenever reference is made to a ‘deoxyoligoribonucleotide’ it may comprise the deoxyribonucleosides deoxyadenosine (A), deoxyguanosine (G), deoxycytidine (C), thymine (T) or deoxyinosine (I). In a preferred aspect, the EON of the present invention is mostly an oligoribonucleotide that may comprise chemical modifications and, at a few specified positions, deoxyribonucleosides (DNA). When reference is made to nucleotides in the oligonucleotide construct, such as cytosine, then 5-methylcytosine, 5-hydroxymethylcytosine, Pyrrolocytidine, and 3-D-Glucosyl-5-hydroxy-methylcytosine are included. When reference is made to adenine, then 2-aminopurine, 2,6-diaminopurine, 3-deazaadenosine, 7-deazaadenosine, 8-azidoadenosine, 8-methyladenosine, 7-aminomethyl-7-deazaguanosine, 7-deazaguanosine, N6-Methyladenine and 7-methyladenine are included. When reference is made to uracil, then 5-methoxyuracil, 5-methyluracil, dihydrouracil, pseudouracil, and thienouracil, dihydrouracil, 4-thiouracil and 5-hydroxymethyluracil are included. When reference is made to guanosine, then 7-methylguanosine, 8-aza-7-deazaguanosine, thienoguanosine and 1-methylguanosine are included. When reference is made to nucleosides or nucleotides, then ribofuranose derivatives, such as 2′-deoxy, 2′-hydroxy, 2-fluororibose and 2′-O-substituted variants, such as 2′-O-methyl, are included, as well as other modifications, including 2′-4′ bridged variants.

The term “comprising” encompasses “including” as well as “consisting of”, e.g. a composition ‘comprising X’ may consist exclusively of X or may include something additional, e.g. X+Y. The term ‘about’ in relation to a numerical value x is optional and means, e.g. x+10%. The word ‘substantially’ does not exclude ‘completely’, e.g. a composition which is ‘substantially free from Y’ may be completely free from Y. Where relevant, the word ‘substantially’ may be omitted from the definition of the invention. The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand. References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity. The term ‘mismatch’ is used herein to refer to opposing nucleotides in a double stranded RNA complex which do not form perfect base pairs according to the Watson-Crick base pairing rules. Mismatch base pairs are G-A, C-A, U-C, A-A, G-G, C-C, U-U base pairs. In some embodiments EONs of the present invention comprise 0, 1, 2 or 3 mismatches, wherein a single mismatch may comprise several sequential nucleotides. In some embodiments EONs of the present invention comprise 0, 1, 2 or 3 wobble base pairs. Wobble base pairs are: G-U, I-U, I-A, and I-C base pairs.

The regular internucleosidic linkages between the nucleotides may be altered by mono- or di-thioation of the phosphodiester bonds to yield phosphorothioate esters or phosphorodithioate esters, respectively. Other modifications of the internucleosidic linkages are possible, including amidation and peptide linkers. In a preferred aspect an EON of the present invention has 1, 2, 3, 4 or more phosphorothioate linkages between the most terminal nucleotides of the EON (hence, preferably at both the 5′ and 3′ end), which means that in the case of 4 phosphorothioate linkages, which is a specifically preferred aspect, the ultimate 5 nucleotides are linked accordingly. It will be understood by the skilled person that the number of such linkages may vary on each end, depending on the target sequence, or based on other aspects, such as stability, toxicity and/or efficiency. In one embodiment of the invention, an EON according to the present invention comprises a substitution of one of the non-bridging oxygens in the phosphodiester linkage. This modification slightly destabilizes base-pairing but adds significant resistance to nuclease degradation. The exact chemistries and formats may depend from oligonucleotide construct to oligonucleotide construct and from application to application, and may be worked out in accordance with the wishes and preferences of those of skill in the art. It is believed in the art that four or more consecutive DNA nucleotides in an oligonucleotide create so-called ‘gapmers’ that—when annealed to their RNA cognate sequences—induce cleavage of the target RNA by RNase H. According to the present invention, RNase H cleavage of the target RNA is generally to be avoided as much as possible.

An EON according to the invention is normally longer than 10 nucleotides, preferably more than 11, 12, 13, 14, 15, 16, still more preferably more than 17 nucleotides. In one embodiment the EON according to the invention is longer than 20 nucleotides. The oligonucleotide according to the invention is preferably shorter than 100 nucleotides, still more preferably shorter than 60 nucleotides, still more preferably shorter than 50 nucleotides. In a preferred aspect, the oligonucleotide according to the invention comprises 18 to 70 nucleotides, more preferably comprises 18 to 60 nucleotides, and even more preferably comprises 18 to 50 nucleotides. Hence, in a particularly preferred aspect, the oligonucleotide of the present invention comprises 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. In another preferred aspect, at either end or at both termini of an EON according to the present invention inverted deoxyT or dideoxyT nucleotides may be incorporated.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of hADAR to deaminate adenosines in a non-discriminative manner, reacting with any adenosine it encounters. The specificity of hADAR1 and 2 can be increased by ensuring several mismatches in the dsRNA that presumably help to position the dsRNA binding domains in a way that has not been clearly defined yet. Additionally, the deamination reaction itself can be enhanced by providing an EON that comprises a mismatch opposite the adenosine to be edited. The mismatch is preferably created by providing a targeting portion having a cytidine opposite the adenosine to be edited. As an alternative, also uridines may be used opposite the adenosine, which, understandably, will not result in a ‘mismatch’ because U and A pair. Upon deamination of the adenosine in the target strand, the target strand will obtain an inosine which, for most biochemical processes, is “read” by the cell's biochemical machinery as a G. Hence, after A to I conversion, the mismatch has been resolved, because I is perfectly capable of base pairing with the opposite C in the targeting portion of the oligonucleotide construct according to the invention. After the mismatch has been resolved due to editing, the substrate is released and the oligonucleotide construct-editing entity complex is released from the target RNA sequence, which then becomes available for downstream biochemical processes, such as splicing and translation. The desired level of specificity of editing the target RNA sequence may depend on the application. Following the instructions in the present disclosure, those of skill in the art will be capable of designing the complementary portion of the oligonucleotide according to their needs, and, with some trial and error, obtain the desired result.

The teaching of the present invention can also be used to edit target RNA sequences in cells within a so-called organoid, such as in vitro generated eye cups. The treated cells, in the organoid, or in vivo, or in vitro or in ex vivo situations will generally have a genetic mutation in which an adenosine is targeted. The mutation may be heterozygous or homozygous. The invention will typically be used to modify point mutations, such as N to A mutations, wherein N may be G, C, U (on the DNA level T), preferably G to A mutations, or N to C mutations, wherein N may be A, G, U (on the DNA level T), preferably U to C mutations. Table 1 displays a list of the most relevant pathogenic G>A mutations found in the ABCA4 gene (and reported in the art), all of which may potentially be targeted by an EON according to the present invention. It is noted that not all (potentially possible) mutations are given here, because an EON of the present invention may also be applied to target an adenosine that is not the result of a G>A mutation, but that is part of a premature stop codon, due to the mutation in fact being the appearance of a T or G. For instance, when a wild type TCA codon (serine) is mutated to TGA (stop) due to a C>G mutation, then targeting the A using an EON according to the present invention changes that stop codon to TGG (tryptophan) that may be tolerated in the ABCA4 protein. Another example may be a wild type AAA codon (lysine) that is mutated to TAA (stop) due to an A>T mutation, then targeting the 5′ A using an EON according to the present invention changes that stop codon to GAA (glutamate) that may be tolerated in the ABCA4 protein.

TABLE 1
Most relevant G > A mutations reported in the human
ABCA4 gene that could be altered through RNA editing.

| DNA change | | | |
|---|---|---|---|
| (cDNA) | Protein | Exon | Reported |
| c.5882G>A | p.(Gly1961Glu) | 42 | 672 |
| c.4469G>A | p.(Cys1490Tyr) | 30 | 159 |
| c.6320G>A | p.(Arg2107His) | 46 | 67 |
| c.6089G>A | p.(Arg2030Gln) | 44 | 67 |
| c.161G>A | p.(Cys54Tyr) | 3 | 54 |
| c.6449G>A | p.(Cys2150Tyr) | 47 | 43 |
| c.4919G>A | p.(Arg1640Gln) | 35 | 36 |
| c.5929G>A | p.(Gly1977Ser) | 43 | 25 |
| c.4594G>A | p.(Asp1532Asn) | 31 | 17 |
| c.3323G>A | p.(Arg1108His) | 22 | 17 |
| c.3364G>A | p.(Glu1122Lys) | 23 | 16 |
| c.2564G>A | p.(Trp855*) | 16 | 13 |
| c.71G>A | p.(Arg24His) | 2 | 13 |
| c.1805G>A | p.(Arg602Gln) | 13 | 12 |
| c.194G>A | p.(Gly65Glu) | 3 | 12 |
| c.1988G>A | p.(Trp663*) | 14 | 11 |
| c.1715G>A | p.(Arg572Gln) | 12 | 10 |
| c.2453G>A | p.(Gly818Glu) | 16 | 9 |
| c.1317G>A | p.(Trp439*) | 10 | 8 |
| c.5646G>A | p.(Met1882Ile) | 40 | 8 |
| c.1648G>A | p.(Gly550Arg) | 12 | 8 |
| c.3064G>A | p.(Glu1022Lys) | 21 | 7 |
| c.4316G>A | p.(Gly1439Asp) | 29 | 6 |
| c.122G>A | p.(Trp41*) | 2 | 6 |
| c.2099G>A | p.(Trp700*) | 14 | 6 |
| c.560G>A | p.(Arg187His) | 5 | 6 |
| c.3272G>A | p.(Gly1091Glu) | 22 | 6 |
| c.214G>A | p.(Gly72Arg) | 3 | 6 |
| c.4069G>A | p.(Ala1357Thr) | 27 | 6 |
| c.1293G>A | p.(Trp431*) | 10 | 5 |
| c.6329G>A | p.(Trp2110*) | 46 | 5 |
| c.1958G>A | p.(Arg653His) | 14 | 5 |
| c.5914G>A | p.(Gly1972Arg) | 43 | 5 |
| c.1852G>A | p.(Gln618Arg), | 13 | 5 |
| | p.(Gly618Arg) | | |
| c.6286G>A | p.(Glu2096Lys) | 46 | 5 |
| c.6342G>A | p.(=), p.? | 46 | 5 |
| c.6410G>A | p.(Cys2137Tyr) | 47 | 5 |
| c.4463G>A | p.(Cys1488Tyr) | 30 | 4 |
| c.6220G>A | p.(Gly2074Ser) | 45 | 4 |
| c.4539G>A | p.? | 30 | 4 |
| c.3233G>A | p.(Gly1078Glu) | 22 | 4 |
| c.1853G>A | p.(Gly618Glu) | 13 | 4 |
| c.2552G>A | p.(Gly851Asp) | 16 | 4 |
| c.3380G>A | p.(Gly1127Glu) | 23 | 4 |
| c.3607G>A | p.(Gly1203Arg) | 24 | 4 |
| c.5087G>A | p.(Ser1696Asn) | 36 | 4 |
| c.5881G>A | p.(Gly1961Arg) | 42 | 4 |
| c.6122G>A | p.(Gly2041Asp) | 44 | 4 |
| c.5189G>A | p.(Trp1730*) | 36 | 3 |
| c.93G>A | p.(Trp31*) | 2 | 3 |
| c.2345G>A | p.(Trp782*) | 15 | 3 |
| c.4436G>A | p.(Trp1479*) | 30 | 3 |
| c.4437G>A | | | |
| c.45G>A | p.(Trp15*) | 1 | 3 |
| c.4653G>A | p.(Trp1551*) | 32 | 3 |
| c.4854G>A | p.(Trp1618*) | 35 | 3 |
| c.4872G>A | p.(Trp1624*) | 35 | 3 |
| c.2243G>A | p.(Cys748Tyr) | 15 | 3 |
| c.2291G>A | p.(Cys764Tyr) | 15 | 3 |
| c.2294G>A | p.(Ser765Asn) | 15/16 | 3 |
| c.3106G>A | p.(Glu1036Lys) | 21 | 3 |
| c.3386G>A | p.(Arg1129His) | 23 | 3 |
| c.5657G>A | p.(Gly1886Glu) | 40 | 3 |
| c.66G>A | p.[(Lys21=, ?)], p.? | 1 | 2 |
| c.1719G>A | p.(Met573Ile) | 12 | 2 |
| c.1783G>A | p.(Ala595Thr) | 13 | 2 |
| c.4849G>A | p.(Val1617Met) | 35 | 2 |
| c.123G>A | p.(Trp41*) | 2 | 2 |
| c.1766G>A | p.(Trp589*) | 13 | 2 |
| c.206G>A | p.(Trp69*) | 3 | 2 |
| c.2090G>A | p.(Trp697*) | 14 | 2 |
| c.2565G>A | p.(Trp855*) | 16 | 2 |
| c.3303G>A | p.(Trp1101*) | 22 | 2 |
| c.4224G>A | p.(Trp1408*) | 28 | 2 |
| c.4383G>A | p.(Trp1461*) | 30 | 2 |
| c.5316G>A | p.(Trp1772*) | 38 | 2 |
| c.818G>A | p.(Trp273*) | 7 | 2 |
| c.178G>A | p.(Ala60Thr) | 3 | 2 |
| c.2069G>A | p.(Gly690Asp) | 14 | 2 |
| c.3148G>A | p.(Gly1050Ser) | 21 | 2 |
| c.3449G>A | p.(Cys1150Tyr) | 23 | 2 |
| c.5531G>A | p.(Gly1844Asp) | 39 | 2 |
| c.6230G>A | p.(Arg2077Gln) | 45 | 2 |
| c.4436G>A | p.(Trp1479*) | | 1 |
| c.4667G>A | p.Ser1545_Gln1555del | | 1 |
| c.6147G>A | p.Ser2002Argfs*11 | | 1 |
| c.5243G>A | p.(Gly1748Glu) | | 1 |
| c.53G>A | p.(Arg18Gln) | 1 | 1 |
| c.6317G>A | p.(Arg2106His) | | 1 |
| c.6694G>A | p.(Glu2232Lys) | | 1 |
| c.4553G>A | p.(Ser1518Asn) | | 1 |

In an aspect of the invention is provided a composition comprising at least one EON according to the invention, preferably wherein said composition comprises at least one excipient, and/or wherein said EON comprises at least one conjugated ligand, that may further aid in enhancing the targeting and/or delivery of said composition and/or said EON to a tissue and/or cell and/or into a tissue and/or cell. Compositions as described here are herein referred to as a composition according to the invention. A composition according to the invention can comprise one or more EONs according to the invention. In the context of this invention, an excipient can be a distinct molecule, but it can also be a conjugated moiety. In the first case, an excipient can be a filler, such as starch. In the latter case, an excipient can for example be a targeting ligand that is linked to the EON according to the invention.

In preferred embodiments of this aspects, such compositions can further comprise a cationic amphiphilic compound (CAC) or a cationic amphiphilic drug (CAD). A CAC is generally a lysosomotropic agent and a weak base that can buffer endosomes and lysosomes (Mae et al. J Contr Rel 2009, 134: 221). Compositions that further comprise a CAC preferably have an improved parameter for RNA editing as compared to a similar composition that does not comprise said CAC. Examples of CACs can be found in for example WO 2018/007475 or WO 2018/134310.

In a preferred embodiment, the composition according to the invention is for use as a medicament. The composition according to the invention is then a pharmaceutical composition. A pharmaceutical composition usually comprises a pharmaceutically accepted carrier, diluent and/or excipient. In a preferred embodiment, a composition according to the invention comprises an EON as defined herein and optionally further comprises a pharmaceutically acceptable formulation, filler, preservative, solubilizer, carrier, diluent, excipient, salt, adjuvant and/or solvent. Such pharmaceutically acceptable carrier, filler, preservative, solubilizer, diluent, salt, adjuvant, solvent and/or excipient may for instance be found in Remington: The Science and Practice of Pharmacy (20^{th} edition, Baltimore, Md.; Lippincott, Williams & Wilkins, 2000). The EON according to the invention may possess at least one ionizable group. An ionizable group may be a base or acid and may be charged or neutral. An ionizable group may be present as ion pair with an appropriate counterion that carries opposite charge(s). Examples of cationic counterions are sodium, potassium, cesium, Tris, lithium, calcium, magnesium, trialkylammonium, triethylammonium, and tetraalkylammonium. Examples of anionic counterions are chloride, bromide, iodide, lactate, mesylate, besylate, triflate, acetate, trifluoroacetate, dichloroacetate, tartrate, phosphate, and citrate.

A pharmaceutical composition according to the invention may comprise an aid in enhancing the stability, solubility, absorption, bioavailability, activity, pharmacokinetics, pharmacodynamics, cellular uptake, and/or intracellular trafficking of the EON, in particular an excipient capable of forming complexes, nanoparticles, microparticles, nanotubes, nanoparticles, nanogels, virosomes, exosomes, hydrogels, poloxamers or pluronics, polymersomes, colloids, microbubbles, vesicles, micelles, lipoplexes, and/or liposomes. Examples of nanoparticles include polymeric nanoparticles, (mixed) metal nanoparticles, carbon nanoparticles, gold nanoparticles, lipid nanoparticles, magnetic nanoparticles and peptide nanoparticles, and combinations thereof. An example of the combination of nanoparticles and oligonucleotides is a spherical nucleic acid (SNA; Barnaby et al. Cancer Treat. Res. 2015, 166: 23).

A preferred composition according to the invention comprises at least one excipient that may further aid in enhancing the targeting and/or delivery of the EON to the tissue and/or a cell and or into a tissue and/or a cell. A preferred tissue or cell is a muscle cell, a retinal cell such as a photoreceptor cell or a cell of the RPE layer, a corneal cell, retinal tissue, or corneal tissue. Preferably, an EON according to the present invention is present in a pharmaceutical composition according to the invention that is administered by intravitreal administration (for instance through direct injection of a naked EON using a syringe) to target photoreceptor cells or RPE in the retina, to edit a target adenosine in a human ABCA4 mRNA and/or ABCA4 pre-mRNA that carries a mutation causing Stargardt disease.

Many of the potential excipients are known in the art and may be categorized as a first type of excipient. Examples of first type of excipients include polymers (e.g. polyethyleneimine (PEI), polypropyleneimine (PPI), dextran derivatives, butylcyanoacrylate (PBCA), hexylcyanoacrylate (PHCA), poly(lactic-co-glycolic acid) (PLGA), polyamines (e.g. spermine, spermidine, putrescine, cadaverine), chitosan, poly(amido amines) (PAMAM), poly(ester amine), polyvinyl ether, polyvinyl pyrrolidone (PVP), polyethylene glycol (PEG), cyclodextrins, hyaluronic acid, colominic acid, and derivatives thereof), dendrimers (e.g. poly (amidoamine)), lipids (e.g. 1,2-dioleoyl-3-dimethylammonium propane (DODAP), dioleoyldimethylammonium chloride (DODAC), phosphatidylcholine derivatives (e.g. 1,2-distearoyl-sn-glycero-3-phosphocholine (DSPC)), lyso-phosphatidylcholine derivatives (e.g. 1-stearoyl-2-lyso-sn-glycero-3-phosphocholine (S-LysoPC)), sphingomyelin, 2-(3-bis-(3-aminopropyl)amino) propylamino)-N-ditetradecyl carbamoyl methylacetamide (RPR209120), phosphoglycerol derivatives (e.g. 1,2-dipalmitoyl-sn-glycero-3-phosphoglycerol, sodium salt (DPPG-Na), phosphatidic acid derivatives (e.g. 1,2-distearoyl-sn-glycero-3-phosphatidic acid, sodium salt (DSPA), phosphoethanolamine derivatives (e.g. dioleoyl-L-R-phosphatidylethanolamine (DOPE), 1,2-distearoyl-sn-glycero-3-phospho ethanolamine (DSPE), 2-diphytanoyl-sn-glycero-3-phosphoethanolamine (DPhyPE)), N-(1-(2,3-dioleoyloxy)propyl-N,N,N-trimethylammonium (DOTAP), N-(1-(2,3-dioleyloxy) propyl)-N,N,N-trimethylammonium (DOTMA), 1,3-di-oleoyloxy-2-(6-carboxy-spermyl) propylamid (DOSPER), (1,2-dimyristoyloxypropyl-3-dimethylhydroxyethylammonium (DMRIE), (N1-cholesteryloxycarbonyl-3,7-diazanonane-1,9-diamine (CDAN), dimethyl dioctadecylammonium bromide (DDAB), 1-palmitoyl-2-oleoyl-sn-glycerol-3-phosphocholine (POPC), (b-L-arginyl-2,3-L-diaminopropionic acid N-palmityl-N-oleyl amide trihydrochloride (AtuFECT01), N,N-dimethyl-3-aminopropane derivatives (e.g. 1,2-distearoyl-N,N-dimethyl-3-aminopropane (DSDMA), 1,2-dioleyloxy-N,N-dimethyl-3-aminopropane (DoDMA), 1,2-dilinoleyloxy-N,N-3-dimethylaminopropane (DLinDMA), 2,2-dilinoleyl-4-dimethylaminomethyl (1,3)-dioxolane (DLin-K-DMA), DLinKC2DMA, DLinMC3DMA (MC3), phosphatdidylserine derivatives (1,2-dioleyl-sn-glycero-3-phospho-L-serine, sodium salt (DOPS)), transfection reagents, proteins (e.g. albumin, gelatins, atelocollagen), and linear or cyclic peptides (e.g. protamine, PepFects, NickFects, polyarginine, hexa-arginine, polylysine, polyornithine, CADY, MPG, cell-penetrating peptides (CPPs), cell-translocating peptides (CTPs), targeting peptides, endosomal escape peptides). Carbohydrates and carbohydrate clusters, when used as distinct compounds, are also suitable for use as a first type of excipient.

Another preferred composition according to the invention may comprise at least one excipient categorized as a second type of excipient. A second type of excipient may comprise or contain a conjugate group as described herein to enhance targeting and/or delivery into a tissue and/or cell, as for example retinal (photoreceptor or RPE) or corneal tissue or cell. The conjugate group may display one or more different or identical ligands. Examples of conjugate group ligands are e.g. peptides, vitamin, aptamers, carbohydrates or mixtures of carbohydrates, proteins, small molecules, antibodies, polymers, drugs. Examples of carbohydrate conjugate group ligands are glucose, mannose, fructose, maltose, galactose, N-galactosamine (GalNAc), glucosamine, N-acetylglucosamine, glucose-6-phosphate, mannose-6-phosphate, and maltotriose. A carbohydrate can also be comprised in a carbohydrate cluster portion, such as a GalNAc cluster portion. A carbohydrate cluster portion can comprise a targeting moiety and, optionally, a conjugate linker. In some embodiments, the carbohydrate cluster comprises 1, 2, 3, 4, 5 or 6, or more GalNAc groups. As used herein, “carbohydrate cluster” means a compound having one or more carbohydrate residues attached to a scaffold or linker group (Maier et al. Bioconj Chem 2003, 14: 18) In this context, “modified carbohydrate” means any carbohydrate having one or more chemical modifications relative to naturally occurring carbohydrates. As used herein, “carbohydrate derivative” means any compound which may be synthesized using a carbohydrate as a starting material or intermediate. As used herein, “carbohydrate” means a naturally occurring carbohydrate, a modified carbohydrate, or a carbohydrate derivative. Both types of excipients may be combined into one single composition as identified herein. An example of a trivalent N-acetylglucosamine cluster is described in WO 2017/062862, which also described a cluster of sulfonamide small molecules. An example of a single conjugate of the small molecule sertraline has also been described (Ferres-Coy et al. Mol. Psych. 2016, 21: 328) as well as conjugates of protein-binding small molecules, including ibuprofen (U.S. Pat. No. 6,656,730), spermine (Noir et al. J. Am. Chem. Soc. 2008, 130: 13500), anisamide (Nakagawa J. Am. Chem. Soc. 2010, 132, 8848) and folate (Dahmen Mol. Ther. Nucl. Acids 2012, 1, e7). Examples of lipid conjugates include fatty acids and their derivatives, e.g. palmityl, palmitoyl, stearyl, stearoyl, myristyl, myristoyl, lauryl, lauroyl, arachidony, arachidonoyl, behenyl, behenoyl, lignoceryl, ligonoceroyl, sapienyl, sapienoyl, oleyl, oleoyl, elaidyl, elaidoyl, vaccenyl, vaccenoyl, linoleyl, linoleoyl, EPA, DHA, cholersteryl, steroid, ω-3 fatty acids, ω-6 fatty acids, in which one or more instances of a lipid, or mixed composition of lipids, is conjugated to the oligonucleotide of the invention. Examples of lipid conjugates of oligonucleotides have been described (WO 2019/232255; Biscans J. Control. Rel. 2019, 302, 116; Biscans Nucl. Acids Res. 2019, 47, 1082; Wang Nucl. Acid Ther. 2019, 29, 245). Examples of vitamins used for conjugation are known (Winkler Ther. Deliv. 2013, 4, 791; U.S. Pat. No. 6,127,533). Conjugates of oligonucleotides with aptamers are also known in the art (Zhao Biomaterials 2015, 67, 42).

Antibodies and antibody fragments can also be conjugated to an oligonucleotide of the invention. In a preferred embodiment, an antibody or fragment thereof targeting tissues of specific interest, particularly retinal or and/or corneal tissue, is conjugated to an oligonucleotide of the invention. Examples of such antibodies and/or fragments are for example targeted to CD71 (transferrin receptor; WO 2016/179257; Sugo J. Control. Rel. 2016, 237, 1). Other oligonucleotide conjugates are known to those skilled in the art and have been reviewed by Winkler et al. (Ther. Deliv. 2013, 4, 791, Manoharan Antisense Nucl. Acid Dev 2004, 12, 103) and Ming et al. (Adv. Drug Deliv. Rev. 2015, 87, 81).

The skilled person may select, combine and/or adapt one or more of the above or other alternative excipients and delivery systems to formulate and deliver an EON for use in the present invention.

Compounds that are comprised in a composition according to the invention can also be provided separately, for example to allow sequential administration of the active ingredient of the composition according to the invention. In such a case, the composition according to the invention is a combination of compounds comprising at least one EON according to the invention with or without a conjugated ligand, at least one excipient, and optionally a CAC as described above.

Preferably, the pharmaceutical composition is for intravitreal administration, preferably by direct injection into the vitreous, and is preferably dosed in an amount ranging from 0.05 mg to 5 mg of total EON per eye. The present invention also relates to a pharmaceutical composition according to the invention, wherein the pharmaceutical composition is for direct intravitreal administration by injection and is dosed in an amount ranging from 0.1 to 1 mg of total EON per eye, such as about 0.1, 0.2, 0.3, 0.4, 0.5, 0.6, 0.7, 0.8, 0.9 or 1.0 mg of total EON per eye. Preferred amounts are 80, 160, and 320 μg oligonucleotide per eye, in which the first dose is preferably twice the amount of the second, third, and further doses. Hence, dosing regimens in which the oligonucleotide (or total of nucleotide, when two or more are administered) is administered in a first injection of 160 μg, followed by subsequent injections of 80 μg, or wherein the first injection contains 320 μg oligonucleotide, followed by maintenance subsequent injections of 160 μg oligonucleotide per eye. Depending on clinical outcomes such dosages may be adjusted. The present invention also relates to a viral vector expressing an EON according to the invention. In yet another aspect, the invention relates to an EON according to the invention, a pharmaceutical composition according to the invention, or a viral vector according to the invention, for use as a medicament. In yet another aspect, the invention relates to an EON according to the invention, a pharmaceutical composition according to the invention, or a viral vector according to the invention, for use in the treatment, prevention, or delay of Stargardt disease. In yet another embodiment, the invention relates to the use of an EON according to the invention, a pharmaceutical composition according to the invention, or a viral vector according to the invention, for the treatment, prevention, or delay of Stargardt disease.

The term “pre-mRNA” refers to a non-processed or partly processed precursor mRNA that is synthesized from a DNA template of a cell by transcription, such as in the nucleus.

Improvements in means for providing an individual or a cell, tissue, organ of said individual with an EON according to the invention, are anticipated considering the progress that has already thus far been achieved. Such future improvements may of course be incorporated to achieve the mentioned effect on restructuring of mRNA using a method of the invention. An EON according to the invention can be delivered as is to an individual, a cell, tissue, or organ of said individual. When administering an EON according to the invention, it is preferred that the EON is dissolved in a solution that is compatible with the delivery method. Retina cells can be provided with a plasmid for EON expression by providing the plasmid in an aqueous solution. Alternatively, a preferred delivery method for an EON or a plasmid for EON expression is a viral vector as outlined above, or nanoparticles. Preferably viral vectors or nanoparticles are delivered to retina cells, more preferably photoreceptor cells or cells of the RPE where the dysfunctional ABCA4 protein is present and causes disease. Such delivery to retina cells or other relevant cells may be in vivo, in vitro or ex vivo.

The skilled person may select and adapt any of the above or other commercially available alternative excipients and delivery systems to package and deliver an EON for use in the current invention to deliver it for the prevention, treatment or delay of an ABCA4-variant related disease or condition. “Prevention, treatment or delay of an ABCA4-variant related disease or condition” is herein preferably defined as preventing, halting, ceasing the progression of, or reversing partial or complete visual impairment or blindness, caused by a genetic defect in the ABCA4 gene.

If multiple distinct EONs according to the invention are used, concentration or dose defined herein may refer to the total concentration or dose of all EONs used, or the concentration or dose of each EONs used or added. Therefore, in one embodiment, there is provided a composition wherein each or the total amount of EONs according to the invention used is dosed in an amount ranged from 0.01 and 20 mg/kg, preferably from 0.05 and 20 mg/kg. A suitable intravitreal dose would be between 0.05 mg and 5 mg, preferably between 0.1 and 1 mg per eye, such as about per eye: 0.1, 0.2, 0.3, 0.4, 0.5, 0.6, 0.7, 0.8, 0.9 or 1.0 mg.

A preferred AON according to the invention is for the treatment of an ABCA4-variant related disease or condition of a human individual. Preferably the ABCA4 variant is selected from the group of variants listed in Table 1, more preferably the c.5882G>A mutation in exon 42 of the human ABCA4 gene, although it cannot be excluded that other ABCA4 mutations will also be targeted in the same treatment, using multiple EONs.

In all embodiments of the invention, the term ‘treatment’ is understood to also include the prevention and/or delay of the ABCA4-variant related disease or condition. An individual, which may be treated using an EON according to the invention may already have been diagnosed as having an ABCA4-variant related disease or condition. Alternatively, an individual which may be treated using an EON according to the invention may not have yet been diagnosed as having an ABCA4-variant related disease or condition such as Stargardt disease but may be an individual having an increased risk of developing such disease or condition in the future given his or her genetic background. A preferred individual is a human individual. In a preferred embodiment the ABCA4-variant related disease or condition is Stargardt disease. Accordingly, the invention further provides an EON according to the invention, or a viral vector according to the invention, or a composition according to the invention for use as a medicament, for treating an ABCA4-variant related disease or condition requiring RNA editing of ABCA4 and for use as a medicament for the prevention, treatment or delay of an ABCA4-variant related disease or condition. Each feature of said use has earlier been defined herein.

The invention further provides the use of an EON according to the invention, or of a viral vector according to the invention, or a (pharmaceutical) composition according to the invention for the treatment of an ABCA4-variant related disease or condition requiring RNA editing (or deamination of the specified adenosine) of ABCA4 (pre-) mRNA. In a preferred embodiment, and for all aspects of the invention, the ABCA4-variant related disorder, disease, or condition is caused by the c.5882G>A mutation in the human ABCA4 gene.

The invention further provides the use of an EON according to the invention, or of a viral vector according to the invention, or a composition according to the invention for the preparation of a medicament, for the preparation of a medicament for treating an ABCA4-variant related disease or condition requiring RNA editing of a specified adenosine within the ABCA4 (pre-) mRNA and for the preparation of a medicament for the prevention, treatment or delay of an ABCA4-variant related disease or condition. Therefore, in a further aspect, there is provided the use of an EON, viral vector or composition as defined herein for the preparation of a medicament, for the preparation of a medicament for treating a condition requiring RNA editing of a specified target adenosine of ABCA4 (pre-) mRNA and for the preparation of a medicament for the prevention, treatment or delay of an ABCA4-variant related disease or condition.

A treatment in a use or in a method according to the invention is at least once, lasts one week, one month, several months, 1, 2, 3, 4, 5, 6 years or longer, such as lifelong. It is to be understood that the treatment as disclosed herein does not edit the cell's DNA and the mutated (pre-) mRNA is constantly being produced by the cell and that mutated (pre-) mRNA may require continuous editing to release the disease. Each EON or equivalent thereof as defined herein for use according to the invention may be suitable for direct administration to a cell, tissue and/or an organ in vivo of individuals already affected or at risk of developing ABCA4-variant related disease or condition, and may be administered directly in vivo, ex vivo or in vitro. The frequency of administration of an EON, composition, compound or adjunct compound of the invention may depend on several parameters such as the severity of the disease, the age of the patient, the mutation of the patient, the number of EONs (i.e. dose), the formulation of said EON, the route of administration and so forth. The frequency may vary between daily, weekly, at least once in two weeks, or three weeks or four weeks or five weeks or a longer period. Dose ranges of an EON according to the invention are preferably designed based on rising dose studies in clinical trials (in vivo use) for which rigorous protocol requirements exist. In a preferred embodiment, a concentration of an EON as defined herein, which is ranged from 0.1 nM and 1 μM is used. Preferably, this range is for in vitro use in a cellular model such as retina cells or retinal tissue, preferably photoreceptor cells and/or RPE. More preferably, the concentration used is ranged from 1 to 400 nM, even more preferably from 10 to 200 nM, even more preferably from 50 to 100 nM. If several EONs are used, this concentration or dose may refer to the total concentration or dose of EONs or the concentration or dose of each EON added. In a preferred embodiment, a viral vector, preferably an AAV vector as described earlier herein, as delivery vehicle for a molecule according to the invention, is administered in a dose ranging from 1×10⁹ to 1×10¹⁷ virus particles per injection, more preferably from 1×10¹⁰ to 1×10¹² virus particles per injection. The ranges of concentration or dose of EONs as given above are preferred concentrations or doses for in vivo, in vitro or ex vivo uses. The skilled person will understand that depending on the EONs used, the target cell to be treated, the gene target and its expression levels, the medium used and the transfection and incubation conditions, the concentration or dose of EONs used may further vary and may need to be optimized any further.

The invention further provides a method for RNA editing of ABCA4 pre-mRNA in a cell comprising contacting the cell, preferably a retina cell, more preferably a photoreceptor cells and/or a RPE cell, with an EON according to the invention, or a viral vector according to the invention, or a composition according to the invention. The features of this aspect are preferably those defined earlier herein. Contacting the cell with an EON according to the invention, or a viral vector according to the invention, or a composition according to the invention may be performed by any method known by the person skilled in the art. Use of the methods for delivery of EONs, viral vectors and compositions described herein is included. Contacting may be directly or indirectly and may be in vivo, ex vivo or in vitro.

The invention further provides a method for the treatment of an ABCA4-variant related disease or condition requiring RNA editing of a specified target adenosine of ABCA4 pre-mRNA of an individual in need thereof (e.g. a patient suffering from Stargardt disease), said method comprising contacting a cell, preferably a retina cell, more preferably a photoreceptor cell and/or a cell in the RPE of said individual with an EON according to the invention, or a viral vector according to the invention, or a composition according to the invention, to deaminate a specific adenosine within said pre-mRNA. The features of this aspect are preferably those defined earlier herein. Contacting the cell, preferably a retina cell, more preferably a photoreceptor cell and/or a cell in the RPE with an EON according to the invention, or a viral vector according to the invention, or a composition according to the invention may be performed by any method known by the person skilled in the art. Use of the methods for delivery of EONs, viral vectors and compositions described herein is included. Contacting may be directly or indirectly and may be in vivo, ex vivo or in vitro. Unless otherwise indicated each embodiment as described herein may be combined with another embodiment as described herein.

The sequence information as provided herein should not be so narrowly construed as to require inclusion of erroneously identified bases. The skilled person can identify such erroneously identified bases and knows how to correct for such errors.

All patent and literature references cited in the present specification are hereby incorporated by reference in their entirety.

### EXAMPLES

### Example 1. RNA Editing of the c.5882G>A Mutation in Exon 42 of Human ABCA4 (Pre-) mRNA Using Four EONs in a Biochemical Assay

The inventors decided to determine whether a relatively common mutation causing Stargardt disease (the c.5882G>A mutation in exon 42 of the human ABCA4 gene) could be repaired by an oligonucleotide that is able to recruit ADAR2 (ex vivo, in vitro, and in vivo), without the need of linking the oligonucleotide physically beforehand to an ADAR2 enzyme. The mutation changes a wild type GGA codon (encoding glycine) to GAA (encoding glutamic acid). Deamination of the middle nucleotide, the first adenosine (A) to inosine (I) would result in a reading frame seen as GGA, which is a return to the wild type situation. If both adenosines would be deaminated to inosines, a GGG codon would be generated. That codon also codes for glycine, which is therefore also the wild type situation, on a protein level. So, deamination of the first adenosine, or deamination of the first adenosine+second adenosine in the same reaction would be beneficial. Notably, if only the second adenosine would be deaminated (resulting in GAG; also coding for glutamic acid) there would be no change in the diseased state. Hence, it would be preferred that only the first adenosine would be deaminated in the RNA editing reaction, but deamination of both adenosines would potentially not be harmful, while single deamination of the second adenosine would not change the amino acid (glutamic acid) coming from the already mutated mRNA.

Initial biochemical assays with four different EONs (ABCA4-1, -2, -3, and -4, respectively; for sequences and chemical modifications, see FIG. 1) were performed with saturating full length hADAR2 protein (recombinantly produced; Genscript) at 12 nM and 1 nM concentration of duplex RNA (target RNA:EON ratio 1:6). Target RNA/EON mixtures were heated at 95° C. and slowly cooled down (30 min) to RT to favour intermolecular interactions. The duplex formation was performed in annealing buffer (10 mM Tris-CI pH 7.4; 1 mM EDTA; 100 mM NaCl). The formed duplex RNA was diluted 2× with water after which it was mixed with 10 ng/μL yeast tRNA (Invitrogen), 20 ng/μL poly A RNA (Qiagen), Protease (EDTA-free Protease Inhibitor Cocktail) and RNase Inhibitor (RNasin Ribonuclease Inhibitors) in editing reaction buffer (15 mM Tris-CI pH 7.4; 1.5 mM EDTA; 60 mM KCl; 3 mM MgSO₄; 0.5 mM DTT; 3% glycerol; 0.003% NP-40; 40 mM K-Glutamate). The editing reaction was initiated by addition of hADAR2 protein in the reaction mix (final volume 50 μL). The reaction was run at 37° C. for 50 min max. At eight different in-between timepoints a 5 μL sample was taken. The reaction for each timepoint was stopped by adding the 5 μL sample to 95 μL boiling 3 mM EDTA and incubating at 95° C. to denature the protein. Then, 6 μl of the stopped reaction mixture was used as a template for cDNA synthesis using the Maxima reverse transcriptase kit with primer gBlock PCR REV: 5′-TGG ACC GAC TGG AAA CGT AG-3′ (SEQ ID NO:13) according to the manufacturer's instructions. The total reaction volume was 20 μl and an extension temperature of 62° C. was used.

Products were amplified for pyrosequencing analysis by PCR, using the Amplitaq gold 360 DNA Polymerase kit (Applied Biosystems) according to the manufacturer's instructions, with 1 μl of the cDNA as template. The following primers were used at a concentration of 10 μM: Pyroseq Fwd ABCA4: 5′-ATG ATG ATG TGG CTG AAG AAA GA-3′ (SEQ ID NO:14), and Pyroseq Rev ABCA4 Biotin, 5′-CCC AGT GAG CAT CTT GAA TGT-3′ (SEQ ID NO:15). The latter primer also contains a biotin conjugated to its 5′ end, as required for the automatic processing during the pyrosequencing reactions.

As inosines pair with cytidines during the cDNA synthesis in the reverse transcription reaction, the nucleotides incorporated in the edited positions during PCR will be guanosines. The percentage of guanosine (edited) versus adenosine (unedited) was defined by pyrosequencing. Pyrosequencing of the PCR products and data analysis were performed by the PyroMark Q48 Autoprep instrument (Qiagen) following the manufacturer's instructions, with 10 μl input of the PCR product and 4 μM of sequencing primer ABCA4-Seq2: 5′-CTC CAG CCC AGC AGT-3′ (SEQ ID NO:16). The settings specifically defined for this target RNA strand included two sets of sequence information. The first of these defines the sequence for the instrument to analyse, while the second set defines the order in which the sequencing reagents corresponding to each nucleotide are to be dispensed, and also includes blank controls (i.e. nucleotides that should not be incorporated at that particular position), which is used by the instrument to define the background signal. The analysis performed by the instrument provides the results for the selected nucleotide as a percentage of adenosine and guanosine detected in that position, and the extent of A-to-I editing at a chosen position will therefore be measured by the percentage of guanosine in that position. Pyrosequencing revealed that editing reached up to 140% efficiency (data not shown). If only a single to-be-edited adenosine would be present in a target codon, then efficiency could not go above 100%. This suggests that not only the first adenosine in the target GAA codon was deaminated to inosine, but that also the second adenosine was edited. It cannot be excluded that in some instances the target sequence was only edited at the second adenosine, resulting in GAG, which would not change the diseased state (as outlined above). Nevertheless, the percentages of editing in any case showed that the first adenosine and/or the second adenosine in the target GAA codon was edited, which shows that the inventors were able to target and edit this ABCA4 mutation very efficiently.

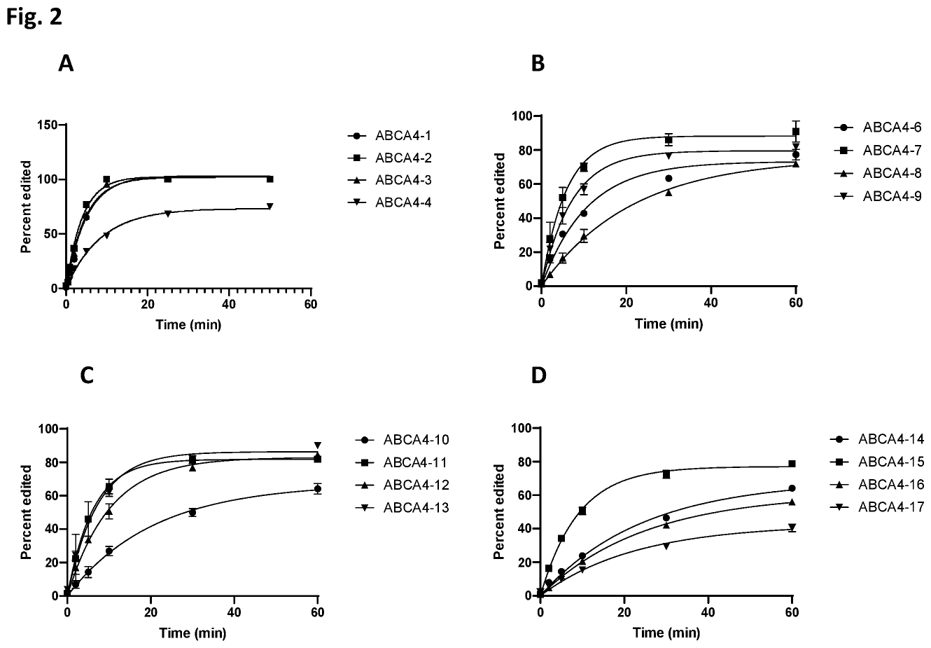

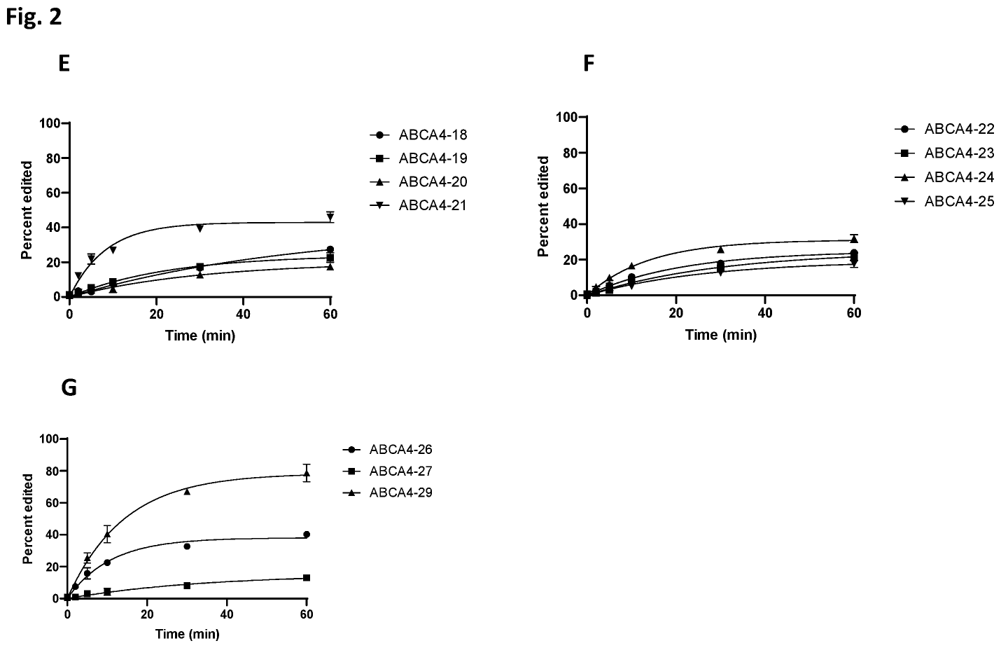

Results of the biochemical experiment with EONs ABCA4-1 to 4 are given in FIG. 2A, which clearly shows that all four tested EONs were able to edit the target adenosine to an inosine, in a relatively rapid fashion. Three EONs (ABCA4-1, -2, and -3) outperformed the ABCA4-4 EON, which is an EON that is almost completely phosphorothioated. This now shows that the inventors were able to target an ABCA4 G>A mutation, allowing the further development for a pharmaceutical compound that is useful for the treatment of Stargardt disease, where the patient suffers from a G>A mutation in the ABCA4 gene.

### Example 2. RNA Editing of the c.5882G>A Mutation in Exon 42 of Human ABCA4 (Pre-) mRNA Using Additional EONs in a Biochemical Assay

In addition to the four initially designed EONs, the inventors designed ABCA4-5 to 29, see FIG. 1 for sequences and modifications. ABCA4-5 contains an internal loop structure, whereas ABCA4-29 contains a variety of additional mismatches with the target sequence. ABCA4-17 to 27 were designed such that they were significantly shorter than the initial EONs and as previously used in the art, while ABCA4-21 to 27 each form a set of split EONs with the ABCA4-28 oligonucleotide that does not target the area of the mutation. The use of short as well as split EONs that work in concert is described in patent application GB 2011428.6 (not published).

The additional set of EONs were also tested in a biochemical assay with a few slight adjustments in comparison to the assay used for the initial four EONs disclosed in Example 1 (for sequences and chemical modifications, see FIG. 1). Saturating full length hADAR2 protein was used (recombinantly produced; Genscript) at 24 nM and 2 nM concentration of duplex RNA (target RNA:EON ratio 1:3). Target RNA/EON mixtures were heated at 95° C. and slowly cooled down (1 h 15 min) to RT to favour intermolecular interactions. The duplex formation was performed in annealing buffer (10 mM Tris-CI pH 7.4; 1 mM EDTA; 100 mM NaCl). The formed duplex RNA was diluted 2× with water and 5× with 0.5× annealing buffer (5 mM Tris-HCl pH 7.4, 0.5 mM EDTA, 50 mM NaCl) after which it was mixed with 10 ng/μL yeast tRNA (Invitrogen), 20 ng/μL poly A RNA (Qiagen), Protease (EDTA-free Protease Inhibitor Cocktail) and RNase Inhibitor (RNasin Ribonuclease Inhibitors) in editing reaction buffer (15 mM Tris-CI pH 7.4; 1.5 mM EDTA; 60 mM KCl; 3 mM MgSO₄; 0.5 mM DTT; 3% glycerol; 0.003% NP-40; 40 mM K-Glutamate). The editing reaction was initiated by addition of hADAR2 protein in the reaction mix (final volume 50 μL). The reaction was run at 37° C. for 60 min max. At six different in-between timepoints a 5 μL sample was taken. The reaction for each timepoint was stopped by adding the 5 μL sample to 95 μL boiling 3 mM EDTA and incubating at 95° C. to denature the protein. Then, 10 μl of the stopped reaction mixture was used as a template for cDNA synthesis using the Maxima reverse transcriptase kit with primer gBlock PCR REV, according to the manufacturer's instructions. The total reaction volume was 20 μl and an extension temperature of 62° C. was used.

Products were amplified for pyromark sequence analysis, using the Amplitaq gold 360 DNA Polymerase kit (Applied Biosystems) according to the manufacturer's instructions, with 1 μl of the cDNA as template. The following primers were used at a concentration of 10 μM: Pyroseq Fwd2 ABCA4: 5′-ACT AAC CAA GAT TTA TCC AGG C-3′ (SEQ ID NO:17) and Pyroseq Rev ABCA4 Biotin (see above). The latter primer also contains a biotin conjugated to its 5′ end, as required for the automatic processing during the pyrosequencing reactions.

Results of the biochemical assays are shown in FIG. 2B to G, each showing the results of 3 or 4 EONs. Clearly, some EONs perform better than others, whereas the shorter versions ABCA4-17, 18, 19, 20, 21, 22, 23, 24, 25, and 27 have lower efficiencies than the longer versions. However, ABCA4-26, which is also a short EON performs very good. ABCA4-29 which has a lot of mismatches with the target sequences does not perform well in comparison to the other EONs, at least in this assay. The biochemical assay was not performed with split EONs (ABCA4-21 to 27, each in concert with ABCA4-28). This was tested in a cell-based assay, see below.

### Example 3. RNA Editing Determined in a Cell Assay

Cell transfection experiments were performed with a variety of different EONs (for sequences and chemical modifications, see FIG. 1). Mouse RPE cells were seeded at 4×10⁴ cells/cm² in DMEM (10% PBS+P/S) in collagen-coated 12-wells plates. After 2-3 hrs incubation at 5% CO₂ and 37° C., the cells were transfected with 500 ng midigene and 500 ng of a plasmid expressing human ADAR2, using Dharmafect duo (Dharmacon) as transfection reagent (3 μL) in a total volume of 1 mL. The midigene is a construct comprising a part of the human ABCA4 genomic sequence, namely intron 40 to intron 43, including the intermediate exons, flanked by Rhodopsin exon 3 and 5 regulated by T7 and preceded by the CMV promoter. Two versions of the midigene were produced, one comprising the wild type ABCA4 sequence, and one comprising the c.5882G>A mutation in exon 42 (mutant). The mutant midigene was co-transfected with the EONs as outlined herein.

After an incubation time of 24 hrs at 5% CO₂ and 37° C., the medium was removed, and cells were washed with PBS. Cells were supplemented with fresh medium (900 μL) and transfected with 100 nM EON using Lipofectamine 2000 (Invitrogen) as transfection reagent. When an EON and a Helper AON (ABCA4-28) was transfected, as a composition comprising split EONs, both oligonucleotides were transfected in an amount of 100 nM. Transfection was performed in a total volume of 1 mL in a ratio of 1 μg EON to 2 μL Lipofectamine 2000. After an incubation time of 6 hrs at 5% CO₂ and 37° C. the medium was removed, cells were washed, and fresh medium was added to the cells.

After an incubation time of in total 24 hrs at 5% CO₂ and 37° C., RNA was isolated from the cells using the RNeasy plus mini kit (Qiagen) and RNA was dissolved in 50 μL RNase free water.

Then, 250 ng of the RNA was used as a template for cDNA synthesis using the Verso cDNA synthesis kit (Thermo scientific) with random hexamers according to the manufacturer's instructions. The total reaction volume was 20 μL. Samples were subsequently used for pyromark sequence analysis and ddPCR analysis.

For pyromark sequence analysis, the products were amplified by using the Amplitaq gold 360 DNA Polymerase kit (Applied Biosystems) according to the manufacturer's instructions, with 5 μl of the cDNA as template. The following primers were used at an end concentration of 0.4 μM: Pyroseq Fwd2 ABCA4 and Pyroseq Rev ABCA4 Biotin (see above). As inosines pair with cytidines during the cDNA synthesis in the reverse transcription reaction, the nucleotides incorporated in the edited positions during PCR will be guanosines. The percentage of guanosine (edited) versus adenosine (unedited) are defined by pyrosequencing. Pyrosequencing of the PCR products and data analysis were performed by the PyroMark Q48 Autoprep instrument (Qiagen) following the manufacturer's instructions, with 10 μl input of the PCR product and 4 μM of the ABCA4-Seq2 primer (SEQ ID NO:16). The settings specifically defined for this target RNA strand included two sets of sequence information. The first of these defines the sequence for the instrument to analyse the difference between guanosine and adenosine in the variable region, while the second set defines the order in which the sequencing reagents corresponding to each nucleotide to be dispensed, and also includes blank controls (i.e. nucleotides that should not be incorporated at that particular position), which is used by the instrument to define the background signal. The analysis performed by the instrument provides the results for the selected nucleotide as a percentage of adenosine and guanosine detected in that position, and the extent of A-to-I editing at a chosen position will therefore be measured by the percentage of guanosine in that position.

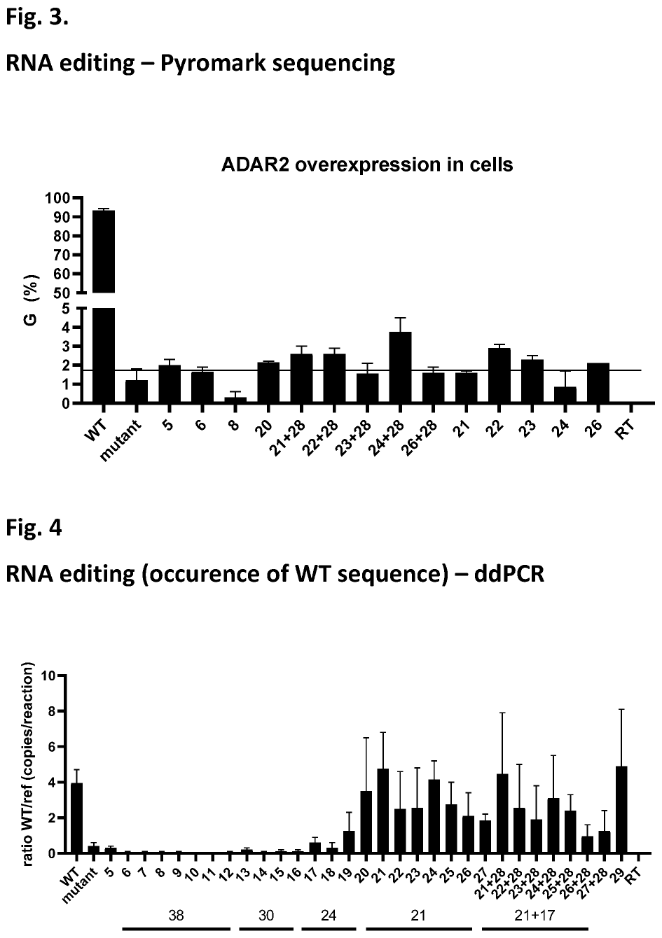

RNA editing results determined through Pyromark sequencing are depicted in FIG. 3, which reveals that many of the tested EONs were in fact able to edit the target adenosine to an inosine in exon 42 of the human ABCA4 (pre-) mRNA, in a cell-based assay, using over-expression of ADAR2.

Then, the wildtype (WT) and reference (ref) copies in the samples were measured using a duplex ddPCR assay. The WT assay was specifically designed for ABCA4 exon 42 G detection (from ABCA4 exon 41 to exon 43) and results in FAM labelled positive droplets. The ref assay was designed to detect reference exon Rho3 to ABCA4 exon 41 and results in HEX labelled positive droplets.

Each ddPCR sample contained 1× ddPCR supermix for probes (no dUTP) (from Biorad), 0.6 μM of each primer (forward ref primer seq: 5′-TTC TGC TAT GGG CAG CTC-3′ (SEQ ID NO:18); reverse ref primer seq: 5′-TGT CTT TCT TCA GCC ACA TC-3′ (SEQ ID NO:19); forward WT primer seq: 5′-CTA ACC AAG ATT TAT CCA GGC-3′ (SEQ ID NO:20); reverse WT primer seq: 5′-CCT GAG GTC ACT GTG GT-3′ (SEQ ID NO:21)), 0.6 μM of each double quenched probe (ref probe seq: 5′-HEX-CAC CGT CAA-ZEN-GGA GGA TTG CCG-IABkFQ-3′ (SEQ ID NO:22); WT probe seq: 5′-6-FAM-TGT GTC GGA-ZEN-GTT CGC CCT GGA GA-IABkFQ-3′ (SEQ ID NO:23)), and 25 ng cDNA in a total volume of 21 μL. Droplets were made from the PCR mixes using the QX200 droplet generator (Biorad). Next, the droplet PCR was performed in a T100 thermal cycler (Biorad) with a heated lid of 105° C. and a ramp temperature of 2° C./sec. The polymerase was heat activated at 95° C. for 10 min. Each cycle the denaturation was performed at 94° C. for 30 sec and the annealing/extension was performed at 60° C. for 30 sec. This was repeated for 40 cycles in total. The enzymes were deactivated at 98° C. for 10 min and the reaction was held at 4° C. Fluorescent signal from the droplets was measured by the QX200 droplet reader (Biorad). Absolute quantification was performed with QuantaSoft software (Bio-Rad). Fluorescence thresholds were set to 3534 for FAM signal and 2000 for HEX signal to determine the number of positive droplets. The ratio of WT/ref was calculated using the copies/reaction. Each sample was measured in duplicate. Average values were used in the calculation.

FIG. 4 shows the WT/ref ratio in each sample. It shows that the highest WT/ref ratio is measured in the samples treated with the smaller EONs. Because the shorter EONs were all complementary to internal sequences of exon 42, whereas the longer versions overlapped with the exon/intron boundary, it was suspected that the use of the longer EONs may have caused exon skipping of exon 42, thereby decreasing the signal in the ddPCR, and therefore the level of RNA editing that is being observed. Hence, it was checked whether such exon skipping did in fact occur.

The exon 42 skip assay was specifically designed for ABCA4 exon 42 deletion. The same ddPCR method as described above was performed using forward exon skip primer: 5′-GAT GTG GCT GAA GAA AGA CA-3′ (SEQ ID NO:24), the same reverse primer as WT assay and double quenched probe exon 42 skip: 5′-6-FAM-AAC TAA CCA-ZEN-AGT GCT TTG GCC TCC-IABkFQ-3′ (SEQ ID NO:25). Fluorescence thresholds were set to 4923 for FAM signal and 2000 for HEX signal to determine the number of positive droplets. The ratio of exon 42 skip/ref was calculated using the copies/reaction.

FIG. 5 shows the exon 42 skip/ref ratio in each sample and shows that the highest skip ratio is measured in samples treated with EONs 13-16, which is indicative for the results shown in FIG. 4, where these EONs show relatively low RNA editing of the target adenosine. In contrast, where relatively high and efficient RNA editing was observed with the smaller EONs that are complementary to an internal exon 42 sequence, such exon skipping could hardly be detected. Importantly, when ABCA4-21 to 27 were used (without ABCA4-28), no skipping was observed at all. Hence, it is preferred to use an EON that does not overlap with the 3′ end of exon 42 and the downstream intron, but rather is fully complementary to a sequence that is completely within exon 42, such as those with SEQ ID NO:8 or 9.

These results now clearly show that the inventors were able to provide EONs that were able to edit the target adenosine in exon 42 of the human ABCA4 (pre-) mRNA to an inosine in cells as well as in an in vitro biochemical setting. Interestingly, the experiment in which split EONs ABCA4-24 and ABCA4-28 were used together the percentage of RNA editing was higher than seen with the separate (single) EON transfections, showing that such a set-up may be preferred. Moreover, it is preferred to use the short EONs (ABCA4-17 to 27, and more preferably ABCA4-21 to 27) because these do not cause a detectable level of exon skipping. It is to be understood that exon skipping of exon 42 is not desired. Rather, the exon skipping should not occur, and only specific RNA editing of the target adenosine in exon 42 should occur. It is demonstrated here that such could be achieved. The preferred use of smaller (split) EONs confirms the results obtained as described in GB 2011428.6 (unpublished).

These experiments show that the inventors were able to target a G>A mutation in exon 42 of the human ABCA4 pre-mRNA, allowing the further development for a pharmaceutical compound that is useful for the treatment of Stargardt disease, where the patient suffers from a G>A mutation in the ABCA4 gene.

1. An RNA editing oligonucleotide (EON) capable of forming a double stranded complex with a target RNA molecule, wherein the EON when complexed with the target RNA molecule, is able to recruit and complex with an ‘Adenosine Deaminase Acting on RNA’ (ADAR) enzyme, thereby allowing the deamination by the ADAR enzyme of a target adenosine in the target RNA molecule, wherein the EON does not form an internal loop structure and wherein the target RNA molecule is a human ABCA4 pre-mRNA or mRNA, or a part thereof.

2. The EON according to claim 1, wherein the EON comprises a Central Triplet of three sequential nucleotides, wherein the nucleotide directly opposite the target adenosine is the middle nucleotide of the Central Triplet and is a cytidine.

3. The EON according to claim 1 or 2, wherein the ADAR enzyme is ADAR2.

4. The EON according to any one of claims 1 to 3, wherein one, two or three nucleotides in the Central Triplet comprise a modification, with the proviso that the middle nucleotide does not have a 2′-O-methyl (2′-OMe) or a 2′-methoxyethoxy (2′-MOE) modification in the sugar moiety.

5. The EON according to claim 4, wherein the modification is selected from the group consisting of deoxyribose (DNA), Unlocked Nucleic Acid (UNA) and 2′-fluororibose.

6. The EON according to any one of claims 1 to 5, wherein the EON comprises at least one non-naturally occurring internucleoside linkage modification selected from the group consisting of: phosphorothioate, chirally pure phosphorothioate, Rp phosphorothioate, Sp phosphorothioate, phosphorodithioate, phosphonoacetate, thophosphonoacetate, phosphonacetamide, thiophosphonacetamide, phosphorothioate prodrug, S-alkylated phosphorothioate, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphorylguanidine, methyl sulfonylphosphoroamidate, phosphoramidite, phosphonamidite, N3′→P5′ phosphoramidate, N3′→P5′ thiophosphoramidate, phosphorodiamidate, phosphorothiodiamidate, sulfamate, dimethylenesulfoxide, sulfonate, triazole, oxalyl, carbamate, methyleneimino, thioacetamido, and their derivatives.

7. The EON according to claim 6, wherein the two, three, four, five, or six terminal nucleotides of the 5′ and 3′ terminus of the EON are linked with phosphorothioate linkages, preferably wherein the terminal five nucleotides at the 5′ and 3′ terminus are linked with phosphorothioate linkages.

8. The EON according to any one of claims 1 to 7, wherein one or more nucleotides in the EON outside the Central Triplet comprise a mono- or disubstitution at the 2′, 3′ and/or 5′ position of the sugar, selected from the group consisting of: —OH; —F; substituted or unsubstituted, linear or branched lower (CI-C10) alkyl, alkenyl, alkynyl, alkaryl, allyl, or aralkyl, that may be interrupted by one or more heteroatoms; O-, S-, or N-alkyl; O-, S-, or N-alkenyl; O-, S-, or N-alkynyl; O-, S-, or N-allyl; O-alkyl-O-alkyl; -methoxy; -aminopropoxy; -methoxyethoxy; -dimethylamino oxyethoxy; and -dimethylaminoethoxyethoxy.

9. The EON according to any one of claims 1 to 8, wherein the target adenosine is part of a premature stop codon in the human ABCA4 pre-mRNA or mRNA.

10. The EON according to any one of claims 1 to 8, wherein the target adenosine is any one of the G>A mutations as provided in Table 1, preferably the c.5882G>A mutation in exon 42 of the human ABCA4 gene.

11. The EON according to any one of claims 1 to 10, wherein the EON comprises or consists of the sequence selected from the group consisting of: SEQ ID NO:1, 2, 3, 4, 5, 6, 7, 8, 9, and 11, preferably SEQ ID NO:8 and 9, and more preferably SEQ ID NO:9.

12. A pharmaceutical composition comprising an EON according to any one of claims 1 to 11, and a pharmaceutically acceptable carrier.

13. A composition comprising a set of two single stranded antisense oligonucleotides (AONs), wherein one AON is an EON according to any one of claims 1 to 11, and the other AON is the ‘Helper AON’, for use in the deamination of a target adenosine in a human ABCA4 pre-mRNA or mRNA, or a part thereof, wherein the Helper AON is complementary to a stretch of nucleotides in the human ABCA4 pre-mRNA or mRNA that is separate from the stretch of nucleotides that is complementary to the EON, wherein the Helper AON has a length of 16 to 22 nucleotides and the EON has a length of 16 to 22 nucleotides.

14. A composition according to claim 13, wherein the EON comprises or consists of a sequence of: SEQ ID NO:9, and wherein the Helper AON comprises or consists of a sequence according to SEQ ID NO:10.

15. An oligonucleotide according to any of claims 1 to 11, or a composition according to any one of claims 12 to 14 for use in the treatment of Stargardt disease.

16. A method for the deamination of at least one specific target adenosine present in a target RNA molecule in a cell, wherein the target RNA molecule is a human ABCA4 pre-mRNA or mRNA, or a part thereof, the method comprising the steps of:
(i) providing the cell with an EON according to any one of claims 1 to 11;
(ii) allowing uptake by the cell of the EON;
(iii) allowing annealing of the EON to the target RNA molecule;
(iv) allowing a mammalian ADAR enzyme comprising a natural dsRNA binding domain as found in the wild type enzyme to deaminate the target adenosine in the target RNA molecule to an inosine; and
(v) optionally identifying the presence of the inosine in the target RNA molecule.

17. The method of claim 14, wherein step (v) comprises:
a) sequencing the target RNA molecule;
b) assessing the presence of a functional, elongated, full length and/or wild type ABCA4 protein;
c) assessing whether splicing of the pre-mRNA was modulated by the deamination; or
d) using a functional read-out, wherein the target RNA after the deamination encodes a functional, full length, elongated and/or wild type ABCA4 protein.


SEQUENCE LISTING (25 sequences)

SEQ ID NO: 1 - DNA, 38 bp, Artificial sequence
  ABCA4-1 EON
guacccaccucuccagggcgaactctgacacacagccu

SEQ ID NO: 2 - DNA, 46 bp, Artificial sequence
  ABCA4-2 EON
ucugcagaguacccaccucuccagggcgaactcagacacacagccu

SEQ ID NO: 3 - DNA, 38 bp, Artificial sequence
  ABCA4-3 EON
guacccaccucuccagggcgaactcagacacacagccu

SEQ ID NO: 4 - DNA, 38 bp, Artificial sequence
  ABCA4-4 EON
guacccacctctccagggcgaactcagacacacagccu

SEQ ID NO: 5 - DNA, 68 bp, Artificial sequence
  ABCA4-5
gguggaauaguauaacaauaugcuaaauguuguuauaguaucccacccgaacucaagacacacagccu

SEQ ID NO: 6 - DNA, 38 bp, Artificial sequence
  ABCA4-6, 7, 8, 9, 10, 11, and 12
ccaaagcacucuccagggcgaacucagacacacagccu

SEQ ID NO: 7 - DNA, 30 bp, Artificial sequence
  ABCA4-13, 14, 15, and 16
cucuccagggcgaacucagacacacagccu

SEQ ID NO: 8 - DNA, 24 bp, Artificial sequence
  ABCA4-17, 18, 19, and 20
agggcgaacucagacacacagccu

SEQ ID NO: 9 - DNA, 21 bp, Artificial sequence
  ABCA4-21, 22, 23, 24, 25, 26, and 27
gcgaacucagacacacagccu

SEQ ID NO: 10 - DNA, 17 bp, Artificial sequence
  ABCA4-28
ccaaagcacucuccagg

SEQ ID NO: 11 - DNA, 38 bp, Artificial sequence
  ABCA4-29
ccacagcacucaccauggcgaacucagacacacagccu

SEQ ID NO: 12 - RNA, 49 nt, Homo sapiens
gacaggcugugugucgaaguucgcccuggagagguggguacucugcaga

SEQ ID NO: 13 - DNA, 20 bp, Artificial sequence
  Primer gBlock PCR REV
tggaccgactggaaacgtag

SEQ ID NO: 14 - DNA, 23 bp, Artificial sequence
  Pyroseq Fwd ABCA4
atgatgatgtggctgaagaaaga

SEQ ID NO: 15 - DNA, 21 bp, Artificial sequence
  Pyroseq Rev ABCA4 Biotin
cccagtgagcatcttgaatgt

SEQ ID NO: 16 - DNA, 15 bp, Artificial sequence
  ABCA4-Seq2 primer
ctccagcccagcagt

SEQ ID NO: 17 - DNA, 22 bp, Artificial sequence
  Pyroseq Fwd2 ABCA4
actaaccaagatttatccaggc

SEQ ID NO: 18 - DNA, 18 bp, Artificial sequence
  Forward ref primer seq
ttctgctatgggcagctc

SEQ ID NO: 19 - DNA, 20 bp, Artificial sequence
  Reverse ref primer seq
tgtctttcttcagccacatc

SEQ ID NO: 20 - DNA, 21 bp, Artificial sequence
  forward WT primer seq
ctaaccaagatttatccaggc

SEQ ID NO: 21 - DNA, 17 bp, Artificial sequence
  reverse WT primer seq
cctgaggtcactgtggt

SEQ ID NO: 22 - DNA, 21 bp, Artificial sequence
  ref probe seq
caccgtcaaggaggattgccg

SEQ ID NO: 23 - DNA, 23 bp, Artificial sequence
  WT probe seq
tgtgtcggagttcgccctggaga

SEQ ID NO: 24 - DNA, 20 bp, Artificial sequence
  forward exon skip primer
gatgtggctgaagaaagaca

SEQ ID NO: 25 - DNA, 24 bp, Artificial sequence
  reverse exon skip primer
aactaaccaagtgctttggcctcc